(19) United States
(12) Patent Application Publication
(10) Pub. No.: US 2020/0060602 A1
(43) Pub. Date: Feb. 27, 2020

(54) MOTION ANALYSIS SYSTEMS AND METHODS OF USE THEREOF

(71) Applicant: Highland Instruments, Somerville, MA (US)

(72) Inventors: Timothy Andrew Wagner, Somerville, MA (US); Laura Dipietro, Cambridge, MA (US); William Edelman, Sharon, MA (US); Seth Elkin-Frankston, Newton, MA (US)

(21) Appl. No.: 16/552,935

(22) Filed: Aug. 27, 2019

Related U.S. Application Data
(63) Continuation of application No. 16/289,279, filed on Feb. 28, 2019.
Application No. 16/289,279 is a continuation of application No. 15/030,451, filed on Apr. 19, 2016, now abandoned.
(60) Provisional application No. 61/903,296, filed on Nov. 12, 2013.

(51) Int. Cl. A61B 5/00; A61B 5/11; A61B 3/113

(52) U.S. Cl. CPC A61B 5/4082; A61B 5/1123; A61B 5/0077; A61B 3/113; A61B 5/1101; A61B 2562/0219; A61B 5/4803; A61B 5/1104; A61B 2576/00; A61B 5/4528; A61B 5/1121; A61B 5/1128

(57) ABSTRACT

The invention generally relates to motion analysis systems and methods of use thereof. In certain aspects, the system includes an image capture device, at least one accelerometer, and a central processing unit (CPU) with storage coupled thereto for storing instructions that when executed by the CPU cause the CPU to receive a first set of motion data from the image capture device related to at least one joint of a subject while the subject is performing a task and receive a second set of motion data from the accelerometer related to the at least one joint of the subject while the subject is performing the task. The CPU also calculates kinematic and/or kinetic information about the at least one joint of a subject from a combination of the first and second sets of motion data, and outputs the kinematic and/or kinetic information for purposes of assessing a movement disorder.

### RELATED APPLICATION

This application is a continuation of U.S. Non-provisional application Ser. No. 16/289,279, filed Feb. 28, 2019, which is a continuation of U.S. Non-provisional application Ser. No. 15/030,451, filed Apr. 19, 2016, which is a 35 U.S.C. § 371 national phase application of PCT/US14/64814, filed Nov. 10, 2014, which claims the benefit of and priority to U.S. provisional application Ser. No. 61/903,296, filed Nov. 12, 2013, the content of each of which is incorporated by reference herein in its entirety.

### GOVERNMENT SUPPORT

This invention was made with government support under 5R44NS080632 awarded by the National Institute of Neurological Disorders and Stroke (NINDS) of the National Institutes of Health (NIH). The government has certain rights in the invention.

### FIELD OF THE INVENTION

The invention generally relates to motion analysis systems and methods of use thereof.

### BACKGROUND

Parkinson's disease (PD) is a chronic and progressive movement disorder. Nearly one million people in the United States are living with Parkinson's disease. Parkinson's disease involves malfunction and death of vital nerve cells in the brain, called neurons. Parkinson's disease affects neurons in an area of the brain known as the substantia nigra. Some of those dying neurons produce dopamine, a chemical that sends messages to the part of the brain that controls movement and coordination. As Parkinson's disease progresses, the amount of dopamine produced in brain areas decreases, leaving a person unable to control movement normally. Parkinson's disease can also be defined as a disconnection syndrome, in which PD-related disturbances in neural connections among subcortical and cortical structures can negatively impact the motor systems of Parkinson's disease patients and further lead to deficits in cognition, perception, and other neuropsychological aspects seen with the disease (Cronin-Golomb Neuropsychology review. 2010; 20(2):191-208. doi: 10.1007/s11065-010-9128-8. PubMed PMID: 20383586; PubMed Central PMCID: PMC2882524).

Numerous rating scales exist for diagnosing Parkinson's disease. The Unified Parkinson's Disease Rating Scale (UPDRS) is the most commonly used scale in the clinical study of Parkinson's Disease. The UPDRS is made up of the following sections: evaluation of Mentation, behavior, and mood; self-evaluation of the activities of daily life (ADLs) including speech, swallowing, handwriting, dressing, hygiene, falling, salivating, turning in bed, walking, cutting food; clinician-scored monitored motor evaluation; Hoehn and Yahr staging of severity of Parkinson disease; and Schwab and England ADL scale.

A problem with the UPDRS is that it is highly subjective because the sections of the UPDRS are evaluated by interview and clinical observation from a team of different specialists. Some sections require multiple grades assigned to each extremity. Because of subjective nature of the UPDRS, it is sometimes difficult to accurately assess a subject. Furthermore, since the UPDRS is based on human observation, it can be difficult to notice subtle changes in disease progression over time. Finally, the nature of UPDRS measurements, based on subjective clinician evaluations, leads to variability due to observer and observer state.

### SUMMARY

The invention provides motion analysis systems that can objectively evaluate a subject for Parkinson's disease, or any type of movement disorder, based on motion data obtained from one or more joints of a subject. Aspects of the invention are accomplished with an image capture device, at least one external body motion sensor, and a computer including processing software that can integrate the data received from the image capture device and the external body motion sensor. Particularly, the processor receives a first set of motion data from the image capture device related to at least one joint of a subject while the subject is performing a task and receives a second set of motion data from the external body motion sensor (e.g., an accelerometer) related to the at least one joint of the subject while the subject is performing the task. The processor then calculates kinematic and/or kinetic information about the at least one joint of the subject from a combination of the first and second sets of motion data, and outputs the kinematic and/or kinetic information for purposes of assessing a movement disorder. In that manner, human observation is removed from the evaluation of a patient, and a standard set of diagnostic measurements is provided for evaluating patients. That provides a unified and accepted assessment rating system across a patient population, which allows for uniform assessment of the patient population. Additionally, since systems of the invention are significantly more sensitive than human observation, subtle changes in disease progression can be monitored and more accurate stratification of a patient population can be achieved. In addition to information from body locations where at least two points of a skeleton (and/or other body tissues) join, joint information can include information from body, body components, and/or limb positions (such as a location on a single skeletal bone, single point of connective tissue, and/or), and/or inferred and/or calculated body positions (such as for example the center of the forearm).

Other types of data can be integrated with systems of the invention to give a fuller picture of a subject. For example, systems of the invention can also include a force plate, which can record balance data of the subject. In such embodiments, the processor receives balance data from the force plate, calculates kinematic and/or kinetic information about the at least one joint of a subject from a combination of the first and second sets of motion data and the balance data, and outputs the kinematic and/or kinetic information for purposes of assessing a movement disorder. Other types of data that are useful to obtain are eye tracking data and voice data. Accordingly, systems of the invention may also include a device for eye tracking and/or a device for voice tracking. In such embodiments, the processor receives balance data, voice data, and/or eye data, calculates kinematic and/or kinetic information about the at least one joint of a subject from a combination of the first and second sets of motion data, the balance data, the eye tracking data, and/or voice data, and outputs the kinematic and/or kinetic information for purposes of assessing a movement disorder. In other embodiments, systems of the invention include a gyroscope and the second set of motion data further includes gyroscopic data. In certain embodiments, the kinematic and/or kinetic information includes information about velocity of the joint.

In certain embodiments, the processor renders received data from the image capture device as a skeletal joint map. In other embodiments, software of the image capture device renders received video data as a skeletal joint map and then sends the skeletal joint map to the processor.

There are any number of tasks that the subject can perform while being evaluated by the motion analysis system. Exemplary tasks include discrete flexion of a limb, discrete extension of a limb, continuous flexion of a limb, continuous extension of a limb, rotation of a limb, opening of a hand, closing of a hand, walking, standing, or any combination thereof.

Typically, the subject is afflicted with a movement disorder. Exemplary movement disorders include diseases which affect a person's control or generation of movement, whether at the site of a joint (e.g., direct trauma to a joint where damage to the joint impacts movement), in neural and/or muscle/skeletal circuits (such as parts of the basal ganglia in Parkinson's Disease), or in both (such as in a certain pain syndrome chronic pain syndrome where for instance a joint could be damaged, generating pain signals, that in turn are associated with change in neural activity caused by the pain). Exemplary movement disorders include Parkinson's disease, Parkinsonism (aka., Parkinsonianism which includes Parkinson's Plus disorders such as Progressive Supranuclear Palsy, Multiple Systems Atrophy, and/or Corticobasal syndrome and/or Cortical-basal ganglionic degeneration), tauopathies, synucleinopathies, Dementia with Lewy bodies, Dystonia, Cerebral Palsy, Bradykinesia, Chorea, Huntington's Disease, Ataxia, Tremor, Essential Tremor, Myoclonus, tics, Tourette Syndrome, Restless Leg Syndrome, Stiff Person Syndrome, arthritic disorders, stroke, neurodegenerative disorders, upper motor neuron disorders, lower motor neuron disorders, muscle disorders, pain disorders, Multiple Sclerosis, Amyotrophic Lateral Sclerosis, Spinal Cord Injury, Traumatic Brain Injury, Spasticity, Chronic Pain Syndrome, Phantom Limb Pain, Pain Disorders, Metabolic Disorders, and/or traumatic injuries.

Another aspect of the invention provides methods for assessing a subject for a movement disorder. Those methods involve receiving a first set of motion data from an image capture device related to at least one joint of a subject while the subject is performing a task, receiving a second set of motion data from an external body motion sensor related to the at least one joint of the subject while the subject is performing the task, calculating, using a computer, kinematic and/or kinetic information about the at least one joint of a subject from a combination of the first and second sets of motion data, and assessing the subject for a movement disorder based on the kinematic and/or kinetic information.

Methods of the invention can additionally include receiving balance data of the subject from a force plate, calculating, using a computer, kinematic and/or kinetic information about the at least one joint of a subject from a combination of the first and second sets of motion data and the balance data, and assessing the subject for a movement disorder based on the kinematic and/or kinetic information. The methods can further involve receiving eye movement data, and/or receiving voice data, which both can be used in the calculation of the kinematic and/or kinetic information.

Systems and methods of the invention can be used in de-novo assessment of a patient for a movement disorder or progression of a movement disorder. Alternatively, systems and methods of the invention can be combined with a stimulation protocol and/or a drug protocol to determine how a subject responds to stimulation. In such embodiments, systems of the invention may involve stimulation apparatuses and methods of the invention may involve providing stimulation to the neural tissue of the subject. The method may be repeated after the subject has received stimulation of their neural tissue, thereby monitoring how a patient has responded to the stimulation they received. That information allows for tuning of subsequent stimulation to better treat the subject.

Since the invention provides new systems for analyzing a subject for a movement disorder, aspects of the invention also provide new methods for assessing whether a subject is afflicted with a movement disorder. For example, another aspect of the invention provides methods of assessing a movement disorder in a subject that involve obtaining a velocity measurement of a joint of a subject while the subject is performing a task, and assessing a movement disorder based on the obtained velocity measurement. Another aspect of the invention provides methods of assessing a movement disorder in a subject that involve obtaining a balance characteristic measurement of a subject using a force plate and an external body motion sensor (e.g., an accelerometer) mounted to the subject while the subject is performing a task, and assessing a movement disorder based on the obtained balance characteristic measurement.

### BRIEF DESCRIPTION OF THE DRAWINGS

FIG. 1 is an illustration showing an embodiment of a motion analysis system of the invention.

FIG. 2 is a flow chart illustrating steps performed by the processor for assessing a movement disorder.

FIG. 3 is an illustration of an exemplary accelerometer useful in the present invention.

FIG. 4 is an illustration of an exemplary gyroscope useful in the present invention.

FIG. 5A is an illustration showing exemplary placement of various components of the external body motion sensor for the hand. FIG. 5B is an illustration showing an alternative exemplary placement of various components of the external body motion sensor for the hand.

FIG. 6A is a graph showing position data recorded from a camera device indicating the position of the wrist in space, provided in X,Y,Z coordinates in the space of the subject, in the units of meters, during a test is provided. The blue line corresponds to the right wrist and the red line to the left wrist. FIG. 6B illustrates information from accelerometers, provided in the X,Y,Z coordinates in the space relative to the accelerometer (i.e., relative to the measurement device) in relative units of the accelerometer. FIG. 6C illustrates information from a gyroscope in relative units of the gyroscope. FIG. 6D illustrates information of the velocity of movement, provided in X,Y,Z coordinates in the space of the subject, with the units of m/s, calculated based on the camera data of the right wrist. FIG. 6E illustrates information of the velocity (red line) based on the camera information in line with the data simultaneously recorded with the accelerometer (blue line). FIG. 6F is a table showing results for a continuous flexion extension task obtained using systems of the invention.

FIG. 7 is a table showing results for a discrete flexion extension task obtained using systems of the invention.

FIG. 8A is a graph showing stability data of the position of the hand. FIG. 8B illustrates peaks of the rotational component of the gyroscope along its X axis that are identified and displayed to the user (blue line in units of the gyroscopic device). The red lines show the triggering device, and the green line demonstrates the peak locations of the movements. FIG. 8C (top half) shows data gathered with the hand held at the shoulder, and FIG. 8C (bottom half) is the same data for the hand held at the waist.

FIG. 9A is a graph showing an example of position data recorded by a camera provided in X,Y,Z coordinates in the space of the subject. The blue line corresponds to the right wrist and the red line to the left wrist. FIG. 9B is a graph showing velocity determined from the camera data (red), accelerometer data (blue line), and the trigger data to mark the beginning of the first and last movement (black lines). The y axis is given in m/s for the velocity data. FIG. 9C is a graph showing data from the power in the movements of the right hand as a function of frequency as determined from the accelerometer data. FIG. 9D is a table showing results obtained using systems of the invention for the task of a subject touching their nose. FIG. 9E is a table showing results obtained using systems of the invention for the task of a subject touching their nose for the purpose of measuring tremor.

FIG. 10A, is a graph showing the weight calculated for the front and back of the left and right foot (in kg). The red line depicts a trigger mark where a clinician has determined a patient has stepped on the board and begun balancing and the second line depicts when the clinician tells the patient the test is over and they can prepare to get off the force plate, the x-axis is in until of time. FIG. 10B is a graph showing a typical examples of data depicting patients center of gravity movements (blue), here depicted in units of length, and area ellipses depicting total movement (red)—the top part shows a patient who has been perturbed (eyes open) and swaying and the bottom part shows a patient standing without perturbation (eyes closed). The time information could be communicated on a third axis or via color coding, here for clarity it is removed in the current depiction. FIG. 10C is a graph showing jerk data, in units of position per time cubed. The top part shows a patient who has been perturbed and swaying (eyes open) and the bottom part shows a patient standing without perturbation (eyes closed). FIG. 10D is a set of two tables showing results. FIG. 10D (top table) shows eyes open and eyes closed data obtained while a subject is standing unperturbed. FIG. 10D (bottom table) shows eyes open data obtained while a subject is being pulled.

FIG. 11A is a graph showing peaks of the rotational component of the gyroscope along its Z axis, identified and displayed to the user (blue line in units of the gyroscopic device). The red lines show the triggering device, and the green line depicts the time instants corresponding to peaks of Z rotational component. The Y-axis is given in the relative units of the gyroscope around its Z-axis, and the X-axis in units of time. The triggering device here is activated on every step. FIG. 11B shows the compiled results of the from the data shown in FIG. 11A, demonstrating the total walk time, and longest time per right step (Peak Distance). FIG. 11C an example of Jerk (the Y-axis is in the units of m/time\hat{ }3, X-axis in terms of time). The blue line corresponds to the period while a person is walking and the open space when the walk and task recording has stopped. FIG. 11D shows the compiled results of the from data shown in FIG. 11C.

FIG. 12A is a table showing results obtained using systems of the invention for a subject performing a continuous flexion extension task. FIG. 12B is a table showing results obtained using systems of the invention for a subject performing a discrete flexion extension task. FIG. 12C is a table showing results obtained using systems of the invention for a subject performing a hand opening and closing task while the arm is positioned at the shoulder. FIG. 12D is a table showing results obtained using systems of the invention for a subject performing a hand opening and closing task while the arm is positioned at the waist. FIG. 12E is a table showing results obtained using systems of the invention for a subject performing the task of touching their nose. FIG. 12F is a table showing results obtained using systems of the invention for a subject performing while the subject is asked to stand still. FIG. 12G is a table showing results obtained using systems of the invention for a subject performing while the subject is walking.

FIG. 13A is a table showing a set of defined criteria for making a differential diagnosis of progressive supranuclear palsy (PSP) compared to other potential movement disorders. FIG. 13B is a table showing symptoms demonstrated in 103 cases progressive supranuclear palsy, in early and later stages, which can be used to make a model for aiding in diagnosing the disease. FIGS. 13C-G are a set of neuro-exam based flow charts based on statistical analysis for diagnosing a movement disorder. FIG. 13E shows a CART analysis of 24 cases of progressive Supranuclear palsy (PSP) and 11 cases of Parkinson's disease (PD). FIG. 13F shows a CART analysis of 24 cases of progressive Supranuclear palsy (PSP) and 14 cases of diffuse Lewy body disease (DLBD). FIG. 13G shows a CART analysis of 24 cases of progressive Supranuclear palsy (PSP) and 11 cases of corticobasal degeneration (CBD).

### DETAILED DESCRIPTION

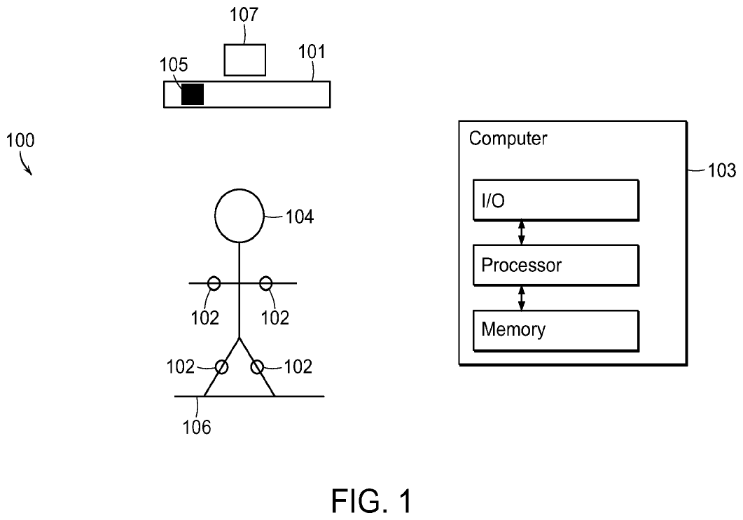

The invention generally relates to motion analysis systems and methods of use thereof. FIG. 1 shows an exemplary motion analysis system 100. The system 100 includes an image capture device 101, at least one external body motion sensor 102, and a central processing unit (CPU) 103 with storage coupled thereto for storing instructions that when executed by the CPU cause the CPU to receive a first set of motion data from the image capture device related to at least one joint of a subject 104 while the subject 104 is performing a task and receive a second set of motion data from the external body motion sensor 102 related to the at least one joint of the subject 104 while the subject 104 is performing the task. The CPU 103 also calculates kinematic and/or kinetic information about the at least one joint of a subject 104 from a combination of the first and second sets of motion data, and outputs the kinematic and/or kinetic information for purposes of assessing a movement disorder. In certain alternative embodiments, more than one image capture device can be used.

Systems of the invention include software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, C#, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, C#, Perl, Java, ActiveX, HTML5, Visual Basic, or JavaScript.

A computer program does not necessarily correspond to a file. A program can be stored in a file or a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification tags or chips, or any other medium which can be used to store the desired information and which can be accessed by a computing device.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

In the exemplary embodiment shown in FIG. 1, system 100 can include a computer 103 (e.g., laptop, desktop, watch, smart phone, or tablet). The computer 103 may be configured to communicate across a network to receive data from image capture device 101 and external body motion sensors 102. The connection can be wired or wireless. Computer 103 includes one or more processors and memory as well as an input/output mechanism(s). In certain embodiments, systems of the invention employ a client/server architecture, and certain processing steps of sets of data may be stored or performed on the server, which may include one or more of processors and memory, capable of obtaining data, instructions, etc., or providing results via an interface module or providing results as a file. Server may be engaged over a network through computer 103.

System 100 or machines according to the invention may further include, for any I/O, a video display unit (e.g., a liquid crystal display (LCD) or a cathode ray tube (CRT)). Computer systems or machines according to the invention can also include an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Memory according to the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

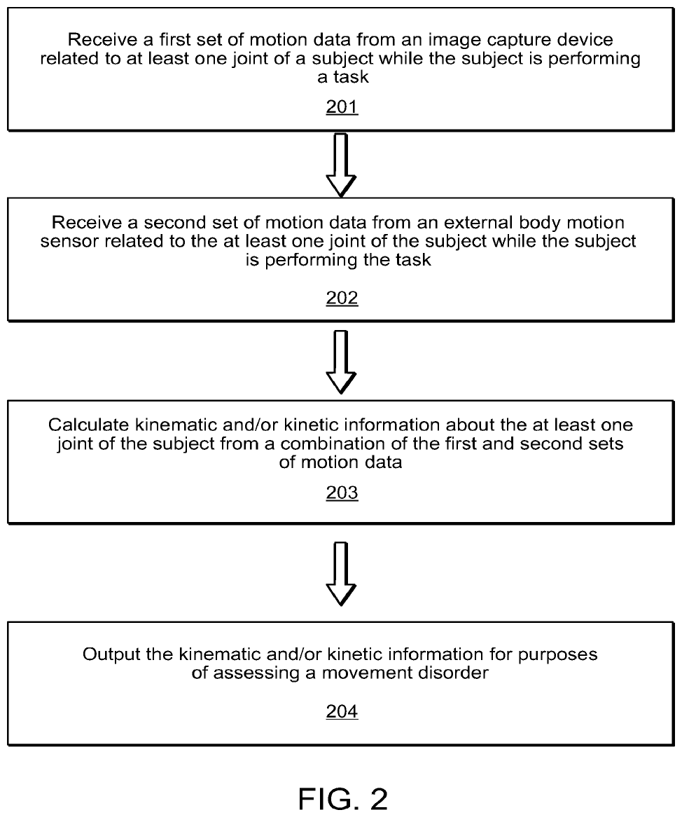

Exemplary step-by-step methods are described schematically in FIG. 2. It will be understood that of the methods described in FIG. 2, as well as any portion of the systems and methods disclosed herein, can be implemented by computer, including the devices described above. At step 201, a first set of motion data from an image capture device is received to the CPU. The first set of motion data is related to at least one joint of a subject while the subject is performing a task. At step 202, a second set of motion data from the external body motion sensor is received to the CPU. The second set of motion data is related to the at least one joint of the subject while the subject is performing the task. In certain embodiments step 201 and step 202 can occur simultaneously in parallel and/or staggered in any order. At step 203, the CPU calculates kinematic and/or kinetic information about the at least one joint of a subject from a combination of the first and second sets of motion data. That calculation can be based on comparing the received data from the subject to a reference set that includes motion data from age and physiologically matched healthy individuals. The reference set of data may be stored locally within the computer, such as within the computer memory. Alternatively, the reference set may be stored in a location that is remote from the computer, such as a server. In that instance, the computer communicates across a network to access the reference set of data. The relative timing of step 201 and step 202 can be controlled by components in measurement devices and/or in the CPU system. At step 204, the CPU outputs the kinematic and/or kinetic information for purposes of assessing a movement disorder.

Additionally, in certain embodiments, patient data can be displayed on a device that the patient can observe (such as on a monitor, a phone, and/or a watch). This data can be used for self-evaluation and/or as part of a training and/or therapeutic regimen. In certain embodiments the data and/or analysis results could be communicated through wired or wireless methods to clinicians who can evaluate the data, such as for example remotely through telemedicine procedures.

### Image Capture Device

Numerous different types of image capture devices can be used in systems of the invention. An exemplary image capture device and its software is described for example in U.S. patent application publication numbers 2010/0199228; 2010/0306716; 2010/0306715; 20100306714; 2010/0306713; 2010/0306712; 20100306671; 20100306261; 2010/0303290; 2010/0302253; 2010/0302257; 2010/0306655; and 2010/0306685, the content of each of which is incorporated by reference herein in its entirety. An exemplary image capture device is the Microsoft Kinect (commercially available from Microsoft).

The image capture device 101 will typically include software for processing the received data from the subject 104 before transmitting the data to the CPU 103. The image capture device and its software enables advanced gesture recognition, facial recognition and optionally voice recognition. The image capture device is able to capture a subject for motion analysis with a feature extraction of one or more joints, e.g., 1 joint, 2 joints, 3 joints, 4 joints, 5 joints, 6 joints, 7 joints, 8 joints, 9 joints, 10 joints, 15 joints, or 20 joints.

In certain embodiments, the hardware of the image capture device includes a range camera that in certain embodiments can interpret specific gestures and/or movements by using an infrared projector and camera. The image capture device may be a horizontal bar connected to a small base with a motorized pivot. The device may include a red, green, and blue (RGB) camera, and depth sensor, which provides full-body 3D motion capture and facial recognition. The image capture device can also optionally include a microphone 105 for capture of sound data (such as for example for voice recordings or for recording sounds from movements). Alternatively, the microphone or similar voice capture device may be separate from the image capture device. The depth sensor may include an infrared laser projector combined with a monochrome CMOS sensor, which captures video data in 3D under any ambient light conditions. The sensing range of the depth sensor is adjustable, and the image capture software is capable of automatically calibrating the sensor based on a subject's physical environment, accommodating for the presence of obstacles. Alternatively, the camera may also capture thermal and/or infrared data. In alternative embodiments sound data can be used for localizing positions, such as would be done in a SONAR method with sonic and/or ultrasonic data. In certain embodiments, the system could employ RAdio Detection And Ranging (RADAR) technology as part of the localizing step.

In certain embodiments, the image capture device is worn on the subject, such as with a GO PRO camera (commercially available from GO Pro). In certain embodiments, the subject wears a light or a light reflecting marker to increase image clarity and/or contrast. In certain embodiments, the system makes use of a camera capable of being attached to the Internet.

The software of the image capture device tracks the movement of objects and individuals in three dimensions. The image capture device and its software uses structured light and machine learning. To infer body position, a two-stage process is employed. First a depth map (using structured light) is computed, and then body position (using machine learning) is inferred.

The depth map is constructed by analyzing a speckle pattern of infrared laser light. Exemplary techniques for constructing such a depth map are described for example in U.S. patent application publication numbers: 2011/0164032; 2011/0096182; 2010/0290698; 2010/0225746; 2010/0201811; 2010/0118123; 2010/0020078; 2010/0007717; and 2009/0185274, the content of each of which is incorporated by reference herein in its entirety. Briefly, the structured light general principle involves projecting a known pattern onto a scene and inferring depth from the deformation of that pattern. Image capture devices described herein uses infrared laser light, with a speckle pattern. The depth map is constructed by analyzing a speckle pattern of infrared laser light. Data from the RGB camera is not required for this process.

The structured light analysis is combined with a depth from focus technique and a depth from stereo technique. Depth from focus uses the principle that objects that are more blurry are further away. The image capture device uses an astigmatic lens with different focal length in x- and y directions. A projected circle then becomes an ellipse whose orientation depends on depth. This concept is further described, for example in Freedman et al. (U.S. patent application publication number 2010/0290698), the content of which is incorporated by reference herein in its entirety.

Depth from stereo uses parallax. That is, if you look at the scene from another angle, objects that are close get shifted to the side more than objects that are far away. Image capture devices used in systems of the invention analyze the shift of the speckle pattern by projecting from one location and observing from another.

Next, body parts are inferred using a randomized decision forest, learned from over many training examples, e.g., 1 million training examples. Such an approach is described for example in Shotten et al. (CVPR, 2011), the content of which is incorporated by reference herein in its entirety. That process starts with numerous depth images (e.g., 100,000 depth images) with known skeletons (from the motion capture system). For each real image, dozens more are rendered using computer graphics techniques. For example, computer graphics are used to render all sequences for 15 different body types, and while varying several other parameters, which obtains over a million training examples.

In the next part of this process, depth images are transformed to body part images. That is accomplished by having the software learn a randomized decision forest, and mapping depth images to body parts. Learning of the decision forest is described in Shotten et al. (CVPR, 2011). In the next part of the process, the body part image is transformed into a skeleton, which can be accomplished using mean average algorithms.

### External Body Motion Sensor

Many types of external body motion sensors are known by those skilled in the art for measuring external body motion. Those sensors include but are not limited to accelerometers, gyroscopes, magnetometers, goniometer, resistive bend sensors, combinations thereof, and the like. In certain embodiments, an accelerometer is used as the external body motion sensor. In other embodiments, a combination using an accelerometer and gyroscope is used. Exemplary external body motion sensors are described for example in U.S. Pat. Nos.: 8,845,557; 8,702,629; 8,679,038; and 8,187,209, the content of each of which is incorporated by reference herein in its entirety. The system of the invention can use one or more external body motion sensors, and the number of sensors used will depend on the number of joints to be analyzed, typically 1 sensor per joint, although in certain embodiments, 1 sensor can analyze more than one joint. For example, one or more joints can be analyzed using one or more sensors, e.g., 1 joint and 1 sensor, 2 joints and 2 sensors, 3 joints and 3 sensors, 4 joints and 4 sensors, 5 joints and 5 sensors, 6 joints and 6 sensors, 7 joints and 7 sensors, 8 joints and 8 sensors, 9 joints and 9 sensors, 10 joints and 10 sensors, 15 joints and 15 sensors, or 20 joints and 20 sensors.

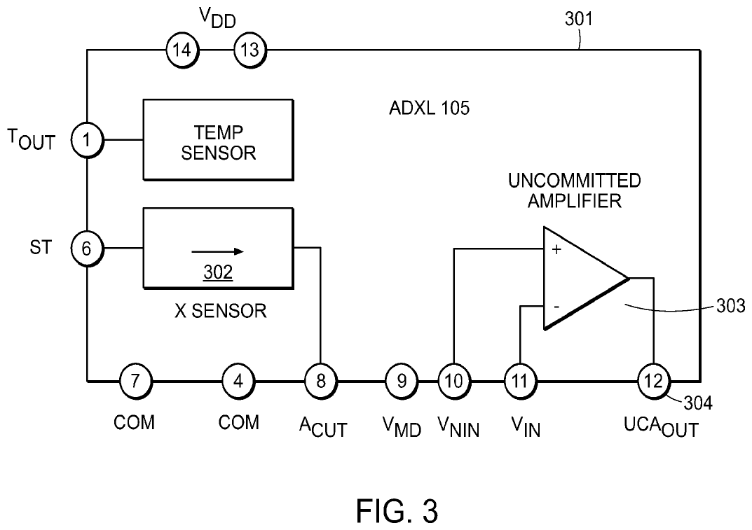

In certain embodiments, external body motion sensor 102 is an accelerometer. FIG. 3 is an electrical schematic diagram for one embodiment of a single axis accelerometer of the present invention. The accelerometer 301 is fabricated using a surface micro-machining process. The fabrication technique uses standard integrated circuit manufacturing methods enabling all signal processing circuitry to be combined on the same chip with the sensor 302. The surface micro-machined sensor element 302 is made by depositing polysilicon on a sacrificial oxide layer that is then etched away leaving a suspended sensor element. A differential capacitor sensor is composed of fixed plates and moving plates attached to the beam that moves in response to acceleration. Movement of the beam changes the differential capacitance, which is measured by the on chip circuitry. All the circuitry 303 needed to drive the sensor and convert the capacitance change to voltage is incorporated on the chip requiring no external components except for standard power supply decoupling. Both sensitivity and the zero-g value are ratiometric to the supply voltage, so that ratiometeric devices following the accelerometer (such as an analog to digital converter (ADC), etc.) will track the accelerometer if the supply voltage changes. The output voltage (VOUT) 304 is a function of both the acceleration input and the power supply voltage (VS).

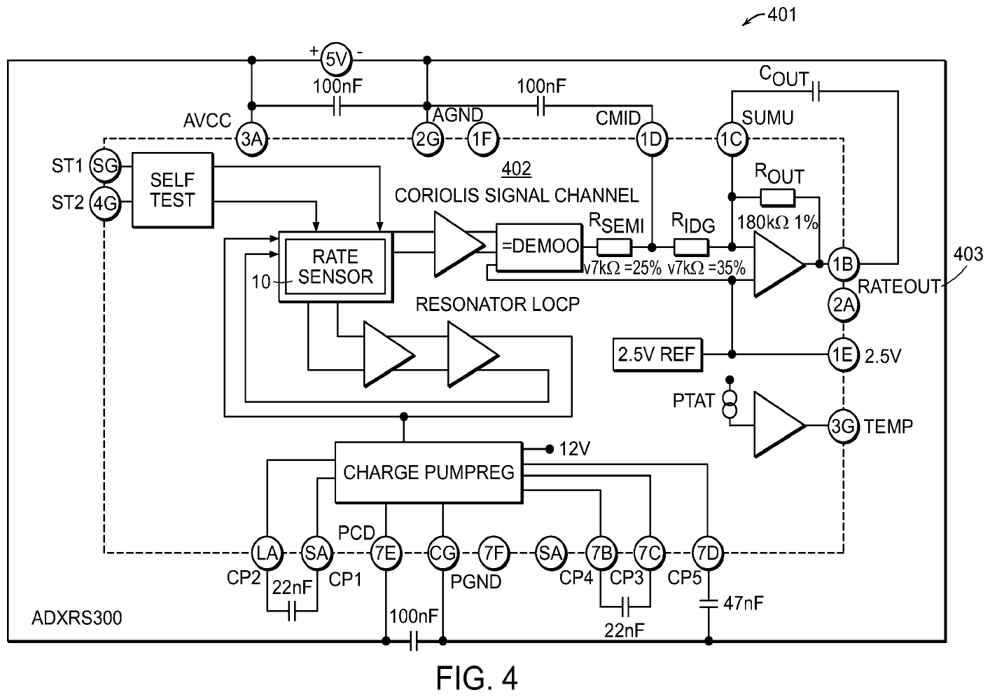

In certain embodiments, external body motion sensor 102 is a gyroscope. FIG. 4 is an electrical schematic diagram for one embodiment of a gyroscope 401 used as a sensor or in a sensor of the present invention. The sensor element functions on the principle of the Coriolis Effect and a capacitive-based sensing system. Rotation of the sensor causes a shift in response of an oscillating silicon structure resulting in a change in capacitance. An application specific integrated circuit (ASIC) 402, using a standard complementary metal oxide semiconductor (CMOS) manufacturing process, detects and transforms changes in capacitance into an analog output voltage 403, which is proportional to angular rate. The sensor element design utilizes differential capacitors and symmetry to significantly reduce errors from acceleration and off-axis rotations.

The accelerometer and/or gyroscope can be coupled to or integrated within into a kinetic sensor board, such as that described in U.S. Pat. No. 8,187,209, the content of which is incorporated by reference herein in its entirety. Therefore, certain embodiments are just an accelerometer and a kinetic sensor board, other embodiments are just a gyroscope and a kinetic sensor board, and still other embodiments are a combination of an accelerometer and a gyroscope and a kinetic sensor board. The kinetic sensor board may include a microprocessor (Texas Instruments mSP430-169) and a power interface section.

The kinetic sensor board and accelerometer and/or gyroscope can be further coupled to or integrated within a transceiver module, such as that described in U.S. Pat. No. 8,187,209, the content of which is incorporated by reference herein in its entirety. The transceiver module can include a blue tooth radio (EB100 A7 Engineering) to provide wireless communications with the CPU 103, and data acquisition circuitry, on board memory, a microprocessor (Analog Devices ADVC7020), and a battery power supply (lithium powered) that supplies power to both the transceiver module and one or more external sensor modules. The transceiver module also includes a USB port to provide battery recharging and serial communications with the CPU 103. The transceiver module also includes a push button input.

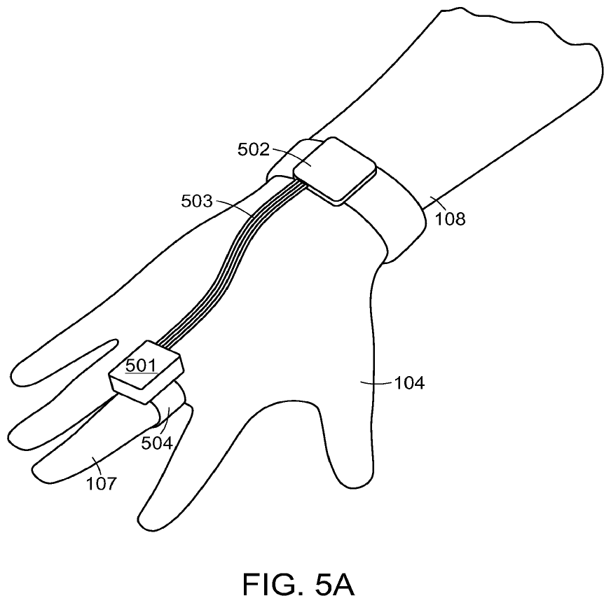

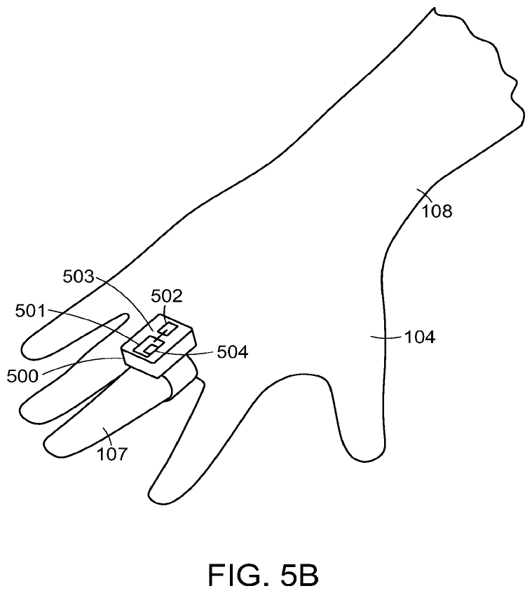

FIG. 5A illustrates one possible embodiment of the subject 104 worn components of the system combining the sensor board 501 and the transceiver module 502. The sensor board 501 consists of at least one accelerometers 504. The sensor board 501 is worn on the subject's 104 finger 106 and the transceiver module 502 is worn on the subject's 104 wrist 108. The transceiver module 502 and one or more external sensor modules 501 are connected by a thin multi-wire leads 503. In an alternative embodiment, the all of the components are made smaller and housed in a single housing chassis 500 that can be mounted on or worn by the subject at one location, say for example all are worn on the finger in a single housing chassis 500, FIG. 5B. In an alternative embodiment, the accelerometer (and/or other motion analysis sensors (e.g., gyroscope)) could be housed in a mobile computing device worn on the subject, such as for example a mobile phone.

In operation, the input to the external sensor module consists of the kinetic forces applied by the user and measured by the accelerometers and/or gyroscopes. The output from the board is linear acceleration and angular velocity data in the form of output voltages. These output voltages are input to the transceiver module. These voltages undergo signal conditioning and filtering before sampling by an analog to digital converter. This digital data is then stored in on board memory and/or transmitted as a packet in RF transmission by a blue tooth transceiver. A microprocessor in the transceiver module controls the entire process. Kinetic data packets may be sent by RF transmission to nearby CPU 103 which receives the data using an embedded receiver, such as blue tooth or other wireless technology. A wired connection can also be used to transmit the data. Alternatively, Kinetic data may also be stored on the on board memory and downloaded to CPU 103 at a later time. The CPU 103 then processes, analyzes, and stores the data.

In certain embodiments, the kinetic sensor board includes at least three accelerometers and measures accelerations and angular velocities about each of three orthogonal axes. The signals from the accelerometers and/or gyroscopes of the kinetic sensor board are preferably input into a processor for signal conditioning and filtering. Preferably, three Analog Devices gyroscopes (ADXRS300) are utilized on the kinetic sensor board with an input range up to 1200 degrees/second. The ball grid array type of component may be selected to minimize size. Additionally, a MEMS technology dual axis accelerometer, from Analog Devices (ADXL210), may be employed to record accelerations along the x and y-axes. Other combinations of accelerometers and gyroscopes known to those skilled in the art could also be used. A lightweight plastic housing may then be used to house the sensor for measuring the subject's external body motion. The external body motion sensor(s) can be worn on any of the subject's joints or in close proximity of any of the subject's joints, such as on the subject's finger, hand, wrist, fore arm, upper arm, head, chest, back, legs, feet and/or toes.

In certain embodiments, the transceiver module contains one or more electronic components such as the microprocessor for detecting both the signals from the gyroscopes and accelerometers. In certain embodiments, the one or more electronic components also filter (and possibly amplify) the kinetic motion signals, and more preferably convert these signals, which are in an analog form into a digital signal for transmission to the remote receiving unit. The one or more electronic components are attached to the subject as part of device or system. Further, the one or more electronic components can receive a signal from the remote receiving unit or other remote transmitters. The one or more electronic components may include circuitry for but are not limited to for example electrode amplifiers, signal filters, analog to digital converter, blue tooth radio, a DC power source and combinations thereof. The one or more electronic components may comprise one processing chip, multiple chips, single function components or combinations thereof, which can perform all of the necessary functions of detecting a kinetic or physiological signal from the accelerometer and/or gyroscope, storing that data to memory, uploading data to a computer through a serial link, transmitting a signal corresponding to a kinetic or physiological signal to a receiving unit and optionally receiving a signal from a remote transmitter. These one or more electronic components can be assembled on a printed circuit board or by any other means known to those skilled in the art. Preferably, the one or more electronic components can be assembled on a printed circuit board or by other means so its imprint covers an area less than 4 in², more preferably less than 2 in², even more preferably less than 1 in², still even more preferably less than 0.5 in², and most preferably less than 0.25 in².

In certain embodiments, the circuitry of the one or more electronic components is appropriately modified so as to function with any suitable miniature DC power source. For example, the DC power source is a battery, such as lithium powered batteries. Lithium ion batteries offer high specific energy (the number of given hours for a specific weight), which is preferable. Additionally, these commercially available batteries are readily available and inexpensive. Other types of batteries include but are not limited to primary and secondary batteries. Primary batteries are not rechargeable since the chemical reaction that produces the electricity is not reversible. Primary batteries include lithium primary batteries (e.g., lithium/thionyl chloride, lithium/manganese dioxide, lithium/carbon monofluoride, lithium/copper oxide, lithium/iodine, lithium/silver vanadium oxide and others), alkaline primary batteries, zinc-carbon, zinc chloride, magnesium/manganese dioxide, alkaline-manganese dioxide, mercuric oxide, silver oxide as well as zinc/air and others. Rechargeable (secondary) batteries include nickel-cadmium, nickel-zinc, nickel-metal hydride, rechargeable zinc/alkaline/manganese dioxide, lithium/polymer, lithium-ion and others. The power system and/or batteries may be rechargeable through inductive means, wired means, and/or by any other means known to those skilled in the art. The power system could use other technologies such as ultra-capacitors.

In certain embodiments, the circuitry of the one or more electronic components comprises data acquisition circuitry. The data acquisition circuitry is designed with the goal of reducing size, lowering (or filtering) the noise, increasing the DC offset rejection and reducing the system's offset voltages. The data acquisition circuitry may be constrained by the requirements for extremely high input impedance, very low noise and rejection of very large DC offset and common-mode voltages, while measuring a very small signal of interest. Additional constraints arise from the need for a “brick-wall” style input protection against ESD and EMI. The exact parameters of the design, such as input impedance, gain and pass-band, can be adjusted at the time of manufacture to suit a specific application via a table of component values to achieve a specific full-scale range and pass-band.

In certain embodiments, a low-noise, lower power instrumentation amplifier is used. The inputs for this circuitry is guarded with preferably, external ESD/EMI protection, and very high-impedance passive filters to reject DC common-mode and normal-mode voltages. Still preferably, the instrumentation amplifier gain can be adjusted from unity to approximately 100 to suit the requirements of a specific application. If additional gain is required, it preferably is provided in a second-order anti-bias filter, whose cutoff frequency can be adjusted to suit a specific application, with due regard to the sampling rate. Still preferably, the reference input of the instrumentation amplifier is tightly controlled by a DC cancellation integrator servo that uses closed-loop control to cancel all DC offsets in the components in the analog signal chain to within a few analog-to digital converter (ADC) counts of perfection, to ensure long term stability of the zero reference.

In certain embodiments, the signals are converted to a digital form. This can be achieved with an electronic component or processing chip through the use of an ADC. More preferably, the ADC restricts resolution to 16-bits due to the ambient noise environment in such chips (other data resolutions can be used such as 8 bit, 32 bit, 64 bit, or more). Despite this constraint, the ADC remains the preferable method of choice for size-constrained applications such as with the present invention unless a custom data acquisition chip is used because the integration reduces the total chip count and significantly reduces the number of interconnects required on the printed circuit board.

In certain embodiments, the circuitry of the sensor board comprises a digital section. More preferably, the heart of the digital section of the sensor board is the Texas Instruments MSP430-169 microcontroller. The Texas Instruments MSP430-169 microcontroller contains sufficient data and program memory, as well as peripherals which allow the entire digital section to be neatly bundled into a single carefully programmed processing chip. Still preferably, the onboard counter/timer sections are used to produce the data acquisition timer.

In certain embodiments, the circuitry of the transceiver module comprises a digital section. More preferably, the heart of the digital section of the sensor board is the Analog Devices ADVC7020 microcontroller. The Analog Devices ADVC7020 microcontroller contains sufficient data and program memory, as well as peripherals which allow the entire digital section to be neatly bundled into a single carefully programmed processing chip. Still preferably, the onboard counter/timer sections are used to produce the data acquisition timer.

In certain embodiments, the circuitry for the one or more electronic components is designed to provide for communication with external quality control test equipment prior to sale, and more preferably with automated final test equipment. In order to supply such capability without impacting the final size of the finished unit, one embodiment is to design a communications interface on a separate PCB using the SPI bus with an external UART and level-conversion circuitry to implement a standard serial interface for connection to a personal computer or some other form of test equipment. The physical connection to such a device requires significant PCB area, so preferably the physical connection is designed to keep the PCB at minimal imprint area. More preferably, the physical connection is designed with a break-off tab with fingers that mate with an edge connector. This allows all required final testing and calibration, including the programming of the processing chip memory, can be carried out through this connector, with test signals being applied to the analog inputs through the normal connections which remain accessible in the final unit. By using an edge fingers on the production unit, and an edge connector in the production testing and calibration adapter, the system can be tested and calibrated without leaving any unnecessary electronic components or too large an PCB imprint area on the final unit.

In certain embodiments, the circuitry for the one or more electronic components comprises nonvolatile, rewriteable memory. Alternatively, if the circuitry for the one or more electronic components doesn't comprise nonvolatile, rewriteable memory then an approach can be used to allow for reprogramming of the final parameters such as radio channelization and data acquisition and scaling. Without nonvolatile, rewriteable memory, the program memory can be programmed only once. Therefore one embodiment of the present invention involves selective programming of a specific area of the program memory without programming the entire memory in one operation. Preferably, this is accomplished by setting aside a specific area of program memory large enough to store several copies of the required parameters. Procedurally, this is accomplished by initially programming the circuitry for the one or more electronic components with default parameters appropriate for the testing and calibration. When the final parameters have been determined, the next area is programmed with these parameters. If the final testing and calibration reveals problems, or some other need arises to change the values, additional variations of the parameters may be programmed. The firmware of various embodiments of the present invention scans for the first blank configuration block and then uses the value from the preceding block as the operational parameters. This arrangement allows for reprogramming of the parameters up to several dozen times, with no size penalty for external EEPROM or other nonvolatile RAM. The circuitry for the one or more electronic components has provisions for in-circuit programming and verification of the program memory, and this is supported by the breakoff test connector. The operational parameters can thus be changed up until the time at which the test connector is broken off just before shipping the final unit. Thus the manufacturability and size of the circuitry for the one or more electronic components is optimized.

In certain embodiments, the circuitry of the one or more electronic components includes an RF transmitter, such as a WiFi based system and/or a blue tooth radio system utilizing the EB100 component from A7 engineering. Another feature of the circuitry of the one or more electronic components preferably is an antenna. The antenna, preferably, is integrated in the rest of the circuitry. The antenna can be configured in a number of ways, for example as a single loop, dipole, dipole with termination impedance, logarithmic-periodic, dielectric, strip conduction or reflector antenna. The antenna is designed to include but not be limited to the best combination of usable range, production efficiency and end-system usability. Preferably, the antenna consists of one or more conductive wires or strips, which are arranged in a pattern to maximize surface area. The large surface area will allow for lower transmission outputs for the data transmission. The large surface area will also be helpful in receiving high frequency energy from an external power source for storage. Optionally, the radio transmissions of the present invention may use frequency-selective antennas for separating the transmission and receiving bands, if a RF transmitter and receiver are used on the electrode patch, and polarization-sensitive antennas in connection with directional transmission. Polarization-sensitive antennas consist of, for example, thin metal strips arranged in parallel on an insulating carrier material. Such a structure is insensitive to or permeable to electromagnetic waves with vertical polarization; waves with parallel polarization are reflected or absorbed depending on the design. It is possible to obtain in this way, for example good cross polarization decoupling in connection with linear polarization. It is further possible to integrate the antenna into the frame of a processing chip or into one or more of the other electronic components, whereby the antenna is preferably realized by means of thin film technology. The antenna can serve to just transfer data or for both transferring data to and for receiving control data received from a remote communication station which can include but is not limited to a wireless relay, a computer or a processor system. Optionally, the antenna can also serve to receive high-frequency energy (for energy supply or supplement). In any scenario, only one antenna is required for transmitting data, receiving data and optionally receiving energy. The couplers being used to measure the radiated or reflected radio wave transmission output. Any damage to the antenna (or also any faulty adaptation) thus can be registered, because it is expressed by increased reflection values.

An additional feature of the present invention is an optional identification unit. By allocating identification codes—a patient code, the remote communication station is capable of receiving and transmitting data to several subjects, and for evaluating the data if the remote communication station is capable of doing so. This is realized in a way such that the identification unit has control logic, as well as a memory for storing the identification codes. The identification unit is preferably programmed by radio transmission of the control characters and of the respective identification code from the programming unit of the remote communication station to the patient worn unit. More preferably, the unit comprises switches as programming lockouts, particularly for preventing unintentional reprogramming.

In any RF link, errors are an unfortunate and unavoidable problem. Analog systems can often tolerate a certain level of error. Digital systems, however, while being inherently much more resistant to errors, also suffer a much greater impact when errors occur. Thus the present invention when used as a digital system, preferably includes an error control sub architecture. Preferably, the RF link of the present invention is digital. RF links can be one-way or two-way. One-way links are used to just transmit data. Two-way links are used for both sending and receiving data.

If the RF link is one-way error control, then this is preferably accomplished at two distinct levels, above and beyond the effort to establish a reliable radio link to minimize errors from the beginning. At the first level, there is the redundancy in the transmitted data. This redundancy is performed by adding extra data that can be used at the remote communication station or at some station to detect and correct any errors that occurred during transit across the airwaves. This mechanism known as Forward Error Correction (FEC) because the errors are corrected actively as the signal continues forward through the chain, rather than by going back to the transmitter and asking for retransmission. FEC systems include but are not limited to Hamming Code, Reed-Solomon and Golay codes. Preferably, a Hamming Code scheme is used. While the Hamming Code scheme is sometimes maligned as being outdated and underpowered, the implementation in certain embodiments of the present invention provides considerable robustness and extremely low computation and power burden for the error correction mechanism. FEC alone is sufficient to ensure that the vast majority of the data is transferred correctly across the radio link. Certain parts of the packet must be received correctly for the receiver to even begin accepting the packet, and the error correction mechanism in the remote communication station reports various signal quality parameters including the number of bit errors which are being corrected, so suspicious data packets can be readily identified and removed from the data stream.

Preferably, at a second, optional level, an additional line of defense is provided by residual error detection through the use of a cyclic redundancy check (CRC). The algorithm for this error detection is similar to that used for many years in disk drives, tape drives, and even deep-space communications, and is implemented by highly optimized firmware within the electrode patch processing circuitry. During transmission, the CRC is first applied to a data packet, and then the FEC data is added covering the data packet and CRC as well. During reception, the FEC data is first used to apply corrections to the data and/or CRC as needed, and the CRC is checked against the message. If no errors occurred, or the FEC mechanism was able to properly correct such errors as did occur, the CRC will check correctly against the message and the data will be accepted. If the data contains residual errors (which can only occur if the FEC mechanism was overwhelmed by the number of errors), the CRC will not match the packet and the data will be rejected. Because the radio link in this implementation is strictly one-way, rejected data is simply lost and there is no possibility of retransmission.

More preferably, the RF link utilizes a two-way (bi-directional) data transmission. By using a two-way data transmission the data safety is significantly increased. By transmitting redundant information in the data emitted by the electrodes, the remote communication station is capable of recognizing errors and request a renewed transmission of the data. In the presence of excessive transmission problems such as, for example transmission over excessively great distances, or due to obstacles absorbing the signals, the remote communication station is capable of controlling the data transmission, or to manipulate on its own the data. With control of data transmission it is also possible to control or re-set the parameters of the system, e.g., changing the transmission channel. This would be applicable for example if the signal transmitted is superimposed by other sources of interference then by changing the channel the remote communication station could secure a flawless and interference free transmission. Another example would be if the signal transmitted is too weak, the remote communication station can transmit a command to increase its transmitting power. Still another example would be the remote communication station to change the data format for the transmission, e.g., in order to increase the redundant information in the data flow. Increased redundancy allows transmission errors to be detected and corrected more easily. In this way, safe data transmissions are possible even with the poorest transmission qualities. This technique opens in a simple way the possibility of reducing the transmission power requirements. This also reduces the energy requirements, thereby providing longer battery life. Another advantage of a two-way, bi-directional digital data transmission lies in the possibility of transmitting test codes in order to filter out external interferences such as, for example, refraction or scatter from the transmission current. In this way, it is possible to reconstruct falsely transmitted data.

Additionally, the external body motion sensor might include code, circuitry, and/or computational components to allow someone to secure (e.g., encrypt, password protect, scramble, etc.) the patient data communicated via wired and/or wireless connections. The code, circuitry, and/or computational components can be designed to match with other components in the system (e.g., camera, eye tracker, voice recorders, balance board, and/or CPU system) that can similarly include code, circuitry, and/or computational components to allow someone to secure (e.g., encrypt, password protect, scramble, etc.) the patient data communicated via wired and/or wireless connections.

### Additional Hardware

In certain embodiments, the motion analysis system 100 includes additional hardware so that additional data sets can be recorded and used in the assessment of a subject for a movement disorder. For example, in certain embodiments, the motion analysis system 100 includes a force plate 106. The subject 104 can stand on the force plate 106 while being asked to perform a task and the force plate 106 will acquire balance data, which can be transmitted through a wired or wireless connection to the CPU 103. An exemplary force plate is the Wii balance board (commercially available from Nintendo). Typically, the force plate will include one or more load sensors. Those sensors can be positioned on the bottom of each of the four legs of the force plate. The sensors work together to determine the position of a subject's center of gravity and to track their movements as they shift your weight from one part of the board to another. Each is a small strip of metal with a sensor, known as a strain gauge, attached to its surface. A gauge consists of a single, long electrical wire that is looped back and forth and mounted onto a hard surface, in this case, the strip of metal. Applying a force on the metal by standing on the plate will stretch or compress the wire. Because of the changes to length and diameter in the wire, its electrical resistance increases. The change in electrical resistance is converted into a change in voltage, and the sensors use this information to figure out how much pressure a subject applied to the plate, as well as the subject's weight.

The sensors' measurements will vary depending on a subject's position and orientation on the plate. For example, if a subject is standing in the front left corner, the sensor in that leg will record a higher load value than will the others. A microcomputer in the plate takes the ratio of the load values to the subject's body weight and the position of the center of gravity to determine the subject's exact motion. That information can then be transmitted to the CPU, through a wireless transmitter in the force plate (e.g., Bluetooth) or a wired connection. In certain embodiments, the individual data recorded from each individual sensor in the force plate can be sent individually to the CPU, or after being processed (in whole or part) within circuitry in the force plate system. In certain embodiments, the system can use digital and/or analog circuitry (such as for example a wheatstone bridge) and/or systems such as those used in digital or analog scales.

The CPU 103 receives the data from the force plate and runs a load detecting program. The load detecting program causes the computer to execute a load value detecting step, a ratio calculating step, a position of the center of gravity calculating step, and a motion determining step. The load value detecting step detects load values put on the support board measured by the load sensor. The ratio calculating step calculates a ratio of the load values detected by the load detecting step to a body weight value of the player. The position of the center of gravity calculating step calculates a position of the center of gravity of the load values detected by the load detecting step. The motion determining step determines a motion performed on the support board by the player on the basis of the ratio and the position of the center of gravity. Alternatively, the force plate can include a processor that performs the above described processing, which processed data is then transmitted to the CPU 103. Alternatively, in certain embodiments only one of the steps is preformed and/or any combination of steps of the load detecting program.

An exemplary force plate and systems and methods for processing the data from the force plate are further described for example in U.S. patent application publication number 2009/0093305, the content of which is incorporated by reference herein in its entirety.

In other embodiments, the motion analysis system 100 includes an eye tracking device 107. FIG. 1 illustrates an exemplary set-up in which the eye tracking device is separate from the image capture device 101. However, in other embodiments, the eye tracking device 107 can be integrated into image capture device 101. Alternatively, a camera component of image capture device 101 can function as eye tracking device 107. A commercially available eye tracking device may be used. Exemplary such devices include ISCAN RK-464 (eye tracking camera commercially available from ISCAN, Inc., Woburn, Mass.), EYELINK II (eye tracking camera commercially available from SR Research Ltd., Ottawa, Canada) or EYELINK 1000 (eye tracking camera commercially available from SR Research Ltd., Ottawa, Canada), or Tobii T60, T120, or X120 (Tobii Technology AB, Danderyd, Sweden). The EYELINK 1000 (eye tracking camera commercially available from SR Research Ltd., Ottawa, Canada) is particularly attractive because subjects do not need to wear any head-mounted apparatus, which is often heavy and bothersome, particularly for young subjects, making tracker calibration a challenge with younger children. Eye-tracker calibration and raw-data processing (e.g., to extract saccades, eliminate blinks, etc.) may be carried out using known techniques. See, e.g., Chan, F., Armstrong, I. T., Pari, G., Riopelle, R. J., and Munoz, D. P. (2005) Saccadic eye movement tasks reveal deficits in automatic response inhibition in Parkinson's disease. Neuropsychologia 43: 784-796; Green, C. R., Munoz, D. P., Nikkei, S. M., and Reynolds, J. N. (2007) Deficits in eye movement control in children with Fetal Alcohol Spectrum Disorders. Alcoholism: Clinical and Exp. Res. 31: 500-511; Peltsch, A., Hoffman, A., Armstrong, I., Pari, G., and Munoz, D. P. (2008) Saccadic impairments in Huntington's disease correlate with disease severity. Exp. Brain Res. (in press); Peters, R. J., Iyer, A., Itti, L., & Koch, C. (2005) Components of bottom-up gaze allocation in natural images. Vision Research, 45(8), 2397-2416, or Tseng et al., U.S. Pat. No. 8,808,195, the content of each of which is incorporated by reference herein in its entirety. In certain embodiments, the camera system 105 can perform aspects of the eye tracking process.

In alternative embodiments, data can be recorded from sound sensors, such as for example voice data. Sound data, such as voice data can be analyzed in many ways, such as for example as a function of intensity, timing, frequency, waveform dynamics, and be correlated to other data recorded from the system. For example, patient data could analyze the power in specific frequency bands that correspond to sounds that are difficult to make during certain movement disorders. In alternative embodiments, the system could use voice recognition so that analysis could be completed by the CPU to determine if a patient could complete cognitive tasks, such as for example remembering words, or to make complex analogies between words. The processes associated with this data could be analog and/or digital (as could all processes throughout this document). In alternative embodiments, the sound sensors could be connected to at least one trigger in the system and/or used as a trigger. See methods examples in: “Digital Signal Processing for Audio Applications” by Anton Kamenov (December 2013); “Speech and Audio Signal Processing: Processing and Perception of Speech and Music” by Ben Gold, Nelson Morgan, Dan Ellis (August 2011); and “Small Signal Audio Design” by Douglas Self (January 2010), the content of each of which is incorporated by reference herein in its entirety. In alternative embodiments, data can be recorded from the eye, such as eye tracking sensors and/or electrooculogram systems. Eye data data can be analyzed in many ways, such as for example eye movement characteristics (e.g., path, speed, direction, smoothness of movements), saccade characteristics, Nystagmus characteristics, blink rates, difference(s) between individual eyes, and/or examples such as those described in, and be correlated to other data recorded from the system. In alternative embodiments, the eye sensors could be connected to at least one trigger in the system and/or used as a trigger. In alternative embodiments, data can be recorded from alternative electrophysiological analysis/recording systems, such as for example EMG or EEG systems.

### Synchronization

The individual component(s) (data acquisition measurement devices (e.g., accelerometer, camera, gyroscope) and/or CPU) of the system can be synchronized via any method known in the field, and communication can take place with wired and/or wireless connections with data that can be of any form, including digital and analog data, and be transmitted uni-directionally and/or bi-directionally (or multi-directionally with multiple components) in any fashion (e.g., serial and/or parallel, continuously and/or intermittently, etc.) during operation. For example, digital information of large data sets can be aligned by synchronizing the first sample and the interval between subsequent samples. Data communicated between at least two devices can be secured (e.g., encrypted), transmitted real-time, buffered, and/or stored locally or via connected media (such as for example for later analysis). In alternative embodiments, the individual components of the system can operate independently and be integrated at a later time point by analyzing the internal clocks of the individual components for offline synchronization. In alternative embodiments, different components and/or sets of components can be synchronized with different methods and/or timings. In certain embodiments, trigger information can be used to mark information about a subject and/or movements that are being assessed by the motion analysis system, such as for example marking when a set of movements of a task begin, and/or marking individual movements in a set of tasks (such as marking each step a patient takes).

Usually two groups of signals will be used in synchronization: timing and triggering (although in certain embodiments, timing can be sufficient). Timing signals usually repeat in a defined, periodic manner and are used as clocks to determine when a single data operation should occur. Triggering signals are stimuli that initiate one or more component functions. Triggering signals are usually single events that are used to control the execution of multiple data operations. The system and/or components can use individual or multiple triggering and/or timing signals.

A variety of timing signals can be used in synchronization. In the simplest form, the individual components of the system run on the same clock(s) (or individual clocks that were synchronized prior to, during, and/or after data acquisition). In alternative methods, additional timing signals can be generated during certain operations of the system, these timing signals could be categorized based on the type of acquisition implemented. For an analog to digital input example, a sample clock in (or connected to) at least one of the data acquisition components of the system controls the time interval between samples, and each time the sample clock ticks (e.g., produces a pulse), one sample (per acquisition channel) is acquired. A Conversion Clock is a clock on or connected to the data acquisition components of the system that directly causes analog to digital conversion. The sampling interval is started on the sample clock tick and then the conversion clock is used to convert the analog signal coming out of a component like a multiplexer on the system with the conversion clock. For counter operations, a counter time-base could be generated from a clock connected to the components of the devices. Triggering signals can be used for numerous functions, such as for example: a start trigger to begin an operation; a pause trigger to pause an ongoing operation; a stop trigger to stop an ongoing operation; or a reference trigger to establish a reference point in an input operation (which could also be used to determine pre-trigger (before the reference) or post-trigger (after the reference) data). Counter output can also be set to re-triggerable so that the specific operation will occur every time a trigger is received.

There are multiple types of synchronization that can be implemented in the system. In general, these types can be abstracted based on the physical level at which they occur. At the lowest level, multi-function synchronization occurs within a single component of the system. The next level is multi-component synchronization which occurs between data acquisition modules (and/or generation tasks (note, the different forms of synchronization can allow for two way communication of data, including both the reading and writing of information during the communication)). Finally, at the highest level multi-group synchronization occurs between groups of components in the system. Multi-group synchronization is the alignment of signals for multiple data acquisition tasks (or generation tasks). This can be accomplished on a single component by routing one signal to the circuitry of different functions, such as analog input, analog output, digital input/output (I/O) and counter/timer operations. Multi-group synchronization is important when trying to synchronize a small number of mixed-signals, such as for example analog data clocked with digital lines, PID control loops, or the frequency response of a system. Multi-component synchronization involves coordinating signals between components. Synchronization between components can use an external connection to share the common signal, but can allow for a high degree of accuracy between measurements on multiple devices. Multi-group synchronization allows multiple sets of components to share at least a single timing and/or triggering signal. This synchronization allows for the expansion of component groups into a single, coordinated structure. Multi-group synchronization can allow for measurements of different types to be synchronized and can be scaled for our system across numerous sets of components. At least one timing or trigger signal can be shared between multiple operations on the same device to ensure that the data is synchronized. These signals are shared by simple signal routing functions that enable built in connections. Further synchronization and communication between components of the system, can be made with any method known in the field, such as for example with methods such as those explained in Data Acquisition Systems: From Fundamentals to Applied Design by Maurizio Di Paolo Emilio (Mar. 22, 2013); Low-Power Wireless Sensor Networks: Protocols, Services and Applications (SpringerBriefs in Electrical and Computer Engineering) by Suhonen, J., Kohvakka, M., Kaseva, V., Hämäläinen, T. D., Hännikäinen, M. (2012); Networking Bible by Barrie Sosinsky (2009); Synchronization Design for Digital Systems (The Springer International Series in Engineering and Computer Science) by Teresa H. Meng (1990); and Virtual Bio-Instrumentation: Biomedical, Clinical, and Healthcare Applications in LabVIEW by Jon B. Olansen (December 2001) which are incorporated by reference herein in their entirety.

### Data Processing

The motion analysis system 100 includes a central processing unit (CPU) 103 with storage coupled to the CPU for storing instructions that when executed by the CPU cause the CPU to execute various functions. Initially, the CPU is caused to receive a first set of motion data from the image capture device related to at least one joint of a subject while the subject is performing a task and receive a second set of motion data from the external body motion sensor related to the at least one joint of the subject while the subject is performing the task. The first and second sets of motion data can be received to the CPU through a wired or wireless connection as discussed above. In certain embodiments, additional data sets are received to the CPU, such as balance data, eye tracking data, and/or voice data. That data can also be received to the CPU through a wired or wireless connection as discussed above.

There are any number of tasks that the subject can perform while being evaluated by the motion analysis system. Exemplary tasks include discrete flexion of a limb, discrete extension of a limb, continuous flexion of a limb, continuous extension of a limb, closing of a hand, opening of a hand, walking, rotation of a joint, holding a joint in a fixed posture (such as to assess tremor while maintaining posture), resting a joint (such as to assess tremor while resting), standing, walking, and/or any combination thereof. Tasks could also include movements which are performed during basic activities of daily living, such as for example walking, buttoning a shirt, lifting a glass, or washing one's self. Tasks could also include movements that are performed during instrumental activities of daily living, which for example could include motions performed during household cleaning or using a communication device. This list of tasks is only exemplary and not limiting, and the skilled artisan will appreciate that other tasks not mentioned here may be used with systems of the invention and that the task chosen will be chosen to allow for assessment and/or diagnosis of the movement disorder being studied. Analysis of the tasks can be made in real time and/or with data recorded by the system and analyzed after the tasks are completed.

Because two different sets of data are being recorded, the CPU includes software and/or hardware for synchronizing data acquisition, such as using methods described above. For example, software on the CPU, can initiate the communication with an image capture device and at least one external patient worn motion sensor. Once the individual components establish a connection (such as for example via a standard handshaking protocol and/or other methods described above), data from all or some of the device components can be recorded in a synchronized manner, and/or stored and/or analyzed by the CPU. The operator can choose to save all or just part of the data as part of the operation. The operator (and/or patient) can initiate a motion analysis system to track a certain task (such as flexing a joint). The initiation (and/or conclusion) of the task can be marked (such as for example by a device which provides a trigger, such as user operated remote control or keyboard, or automatically via software based initiation) on the data that is being recorded by the CPU (and/or in all or some of the individual system components (e.g., an external patient worn motion sensor)) such as could be used for analysis. The data being recorded can be displayed on a computer screen during the task (and/or communicated via other methods, such as for example through speakers if an audio data is being assessed). The data may be stored and analyzed later. The data may be analyzed in real-time, in part or in full, and the results may be provided to the operator and or stored in one of the system components. The data and analysis results could be communicated through wired or wireless methods to clinicians who can evaluate the data, such as example remotely through telemedicine procedures (additionally in certain embodiments the system can be controlled remotely). The process could be run in part or entirely by a patient and/or another operator (such as for example a clinician). In an alternative embodiment, all of the components of the system can be worn, including the image capturing camera, to provide a completely mobile system (the CPU for analysis could be housed on the patient, or the synchronized data could be communicated to an external CPU for all or part of the analysis of the data).

As mentioned above, the system can obtain data from 1 or more joints, e.g., 1 joint, 2 joints, 3 joints, 4 joints, 5 joints, 6 joints, 7 joints, 8 joints, 9 joints, 10 joints, 15 joints, or 20 joints. In certain embodiments, data are recorded with all the sensors, and only the data recorded with the sensors of interest are analyzed. In other embodiments, only data of selected sensors is recorded and analyzed.

The CPU and/or other components of the system are operably linked to at least one trigger, such as those explained above. In alternative embodiments, a separate external component (or an additional integrated system component) can be used to trigger all or some of the components of the system (such as for example one or more remotes of a Wii video gaming system, commercially available from Nintendo, an external computer, a tablet device, an external keyboard, a watch, and/or a mobile phone). In alternative embodiments, the trigger could be voice activated, such as when using a microphone. In alternative embodiments the trigger could be motion activated (such as for example through hand movements, body postures, and/or specific gestures that are recognized). A trigger can mark events into the recorded data, in an online fashion. Additionally, any one of these external devices can be used to write to the data being recorded to indicate when a task is being performed by an individual being evaluated with the system (for example an observer, or individual running the system, while evaluating a patient can indicate when the patient is performing one of the tasks, such as using the device to mark when a flexion and extension task is started and stopped). In certain embodiments the events marked by a trigger can later be used for further data analysis, such as calculating duration of specific movements, or for enabling additional processes such as initiating or directing brain stimulation. In certain embodiments, multiple triggers can be used for functions that are separate or integrated at least in part.

The CPU is then caused to calculate kinematic and/or kinetic information about at least one joint of a subject from a combination of the first and second sets of motion data, which is described in more detail below. Then the CPU is caused to output the kinematic and/or kinetic information for purposes of assessing a movement disorder. Exemplary movement disorders include diseases which affect a person's control or generation of movement, whether at the site of a joint (e.g., direct trauma to a joint where damage to the joint impacts movement), in neural or muscle/skeletal circuits (such as parts of the basal ganglia in Parkinson's Disease), or in both (such as in a certain pain syndrome chronic pain syndrome where for instance a joint could be damaged, generating pain signals, that in turn are associated with change in neural activity caused by the pain). Exemplary movement disorders include Parkinson's disease, Parkinsonism (aka., Parkinsonianism which includes Parkinson's Plus disorders such as Progressive Supranuclear Palsy, Multiple Systems Atrophy, and/or Corticobasal syndrome and/or Cortical-basal ganglionic degeneration), tauopathies, synucleinopathies, Dementia with Lewy bodies, Dystonia, Cerebral Palsy, Bradykinesia, Chorea, Huntington's Disease, Ataxia, Tremor, Essential Tremor, Myoclonus, tics, Tourette Syndrome, Restless Leg Syndrome, Stiff Person Syndrome, arthritic disorders, stroke, neurodegenerative disorders, upper motor neuron disorders, lower motor neuron disorders, muscle disorders, pain disorders, Multiple Sclerosis, Amyotrophic Lateral Sclerosis, Spinal Cord Injury, Traumatic Brain Injury, Spasticity, Chronic Pain Syndrome, Phantom Limb Pain, Pain Disorders, Metabolic Disorders, and/or traumatic injuries.

The data can be used for numerous different types of assessments. In one embodiment, the data is used to assess the effectiveness of a stimulation protocol. In such embodiments, a subject is evaluated with the motion analysis system at a first point in time, which services as the baseline measurement. That first point in time can be prior to receiving any stimulation or at some point after a stimulation protocol has been initiated. The CPU is caused to calculate a first set of kinematic and/or kinetic information about the at least one joint of a subject from a combination of the first and second sets of motion data while the subject is performing a task. That data is stored by the CPU or outputted for storage elsewhere. That first set of kinematic and/or kinetic information is the baseline measurement. The subject is then evaluated with the motion analysis system at a second point in time after having received at least a portion or all of a stimulation protocol. The CPU is caused to calculate a second set kinematic and/or kinetic information about the at least one joint of a subject from a combination of the first and second sets of motion data while the subject is performing a task. That second set of data is stored by the CPU or outputted for storage and/or presentation elsewhere. The first and second sets of data are then compared, either by the CPU or by a physician having received from the CPU the outputted first and second sets of data. The difference, if any, between the first and second sets of data informs a physician as to the effectiveness of the stimulation protocol for that subject. This type of monitoring can be repeated numerous times (i.e., more than just a second time) to continuously monitor the progress of a subject and their response to the stimulation protocol. The data also allows a physician to adjust the stimulation protocol to be more effective for a subject.

In other embodiments, the motion analysis system of the invention is used for initial diagnosis or assessment of a subject for a movement disorder. Such embodiments, use a reference set of data to which a subject is compared in order to make a diagnosis or assessment of the subject. The reference set, stored on the CPU or remotely on a server operably coupled to the CPU, includes data of normal healthy individuals and/or individuals with various ailments of various ages, genders, and/or body type (e.g., height, weight, percent body fat, etc.). Those healthy individuals and/or individuals with various ailments have been analyzed using the motion analysis system of the invention and their data is recorded as baseline data for the reference data set (in alternative embodiments, a reference set can be developed by modeling simulation motion data and/or a reference set could be developed from a model developed based on the analysis of assessments of healthy individuals and/or patients). The reference set of data could be based on previous measurements taken from the patient currently being assessed. A test subject is then evaluated using the motion analysis system of the invention and their kinematic and/or kinetic information is compared against the appropriate population in the reference set, e.g., the test subject data is matched to the data of a population within the reference set having the same or similar age, gender, and body type as that of the subject. The difference, if any, between the test subject's kinematic and/or kinetic information as compared to that of the reference data set allows for the assessment and/or diagnosis of a movement disorder in the subject. Typically, at least a 25% difference (e.g., 30%, 35%, 40%, 45%, 50%, 60%, 70%, 80%, 90%, 95%, 95%, or 99%) between the kinematic and/or kinetic information of the subject and that of the reference data set is an indication that the subject has a movement disorder. The greater the difference between the kinematic and/or kinetic information of the subject and that of the reference data set allows for the assessment of the severity or degree of progression of the movement disorder. For example, a subject with at least 50% difference (e.g., 55%, 60%, 70%, 80%, 90%, 95%, 95%, or 99%) between their kinematic and/or kinetic information and that of the reference set would be considered to have a severe form of the movement disorder. In certain diagnostic tests, just the presence of a characteristic, for example a Babinski sign, would be enough to demonstrate a patient has an ailment, which for a Babinski would demonstrate an upper motor neuron deficit. In certain diagnostic evaluations, smaller differences can be demonstrated and/or tracked, such as for example to track a patients progress to a therapy (such as when comparing a patient's motion analysis results previous motion analysis results from a previous exam of the patient). Furthermore multiple small differences (e.g., less than 25%), for example in at least two criteria, between a tested patient and a data set can be used to make a probabilistic diagnosis that a patient suffers from a disorder (for example, in certain alternative embodiments, multiple changes, with changes as small as 1%, could be used to make a statistical model, that can have a predictive capabilities with high probability that a disease is present (such as for example with 80%, 90%, 95%, 99%, 99.9% and 100% probability)—for example: a statistical model based on 10 different movement task characteristics could be assessed which makes a diagnosis based on a weighted probabilistic model, a disease diagnosis model based on derived results or grouped results (e.g., positive presence of a Babinski sign when 99 other tested criteria were not met, would still be an indication of an upper motor neuron disease), and/or model based on patient history and a result(s) derived from the motion analysis system while patients are performing a movement or set of movement tasks).

Additionally, measures could be used to determine characteristics of movement (such as quality and/or kinematics) at a baseline visit and used to evaluate the impact of a therapy throughout the course a treatment paradigm (eventually the system could be integrated into the therapy providing system to make a closed loop system to help determine or control therapeutic dosing, such as integrating a motion analysis suite with a neurostimulation system (which could further be integrated with other systems, such as computerized neuro-navigation systems and stimulation dose models such as could be developed with finite element models, see for example U.S. patent application publication numbers 2011/0275927 and 2012/0226200, the content of each of which is incorporated by reference herein in its entirety)). A motion analysis suite could further be used to develop new clinical scores based on the quantitative information gathered while evaluating patients (such as for example, one could track Parkinson patients with the system and use the results to come up with a new clinical metric(s) to supplement the UPDRS part III scores for evaluating the movement pathology in the patient).

Certain exemplary embodiments are described below to illustrate the kinematic and/or kinetic information that is calculated for the first and second data sets and the output of that calculation.

In one embodiment, bradykinesia is assessed. A subject is asked to perform 10 arm flexion-extension movements as fast as possible (note that this number (e.g., 10 movements) is just exemplary, and that 1, 2, 3 movements, and so on could be completed. Furthermore, in alternative embodiments, just one movement type (e.g., flexion), any other type of movement(s), and/or groups of movement can be examined. Additionally any joint or group of joints can be assessed. Furthermore, in certain embodiments the tests could be completed with more or less body motion sensors placed at different locations on the body, and/or the analysis can be completed for different joint(s)). Furthermore, during some tests the patient will perform more or less movements than asked (for example, sometimes they are unable to complete all the tasks due to a pathology, other times they might simply loose count of how many movements they have performed). This test can then be repeated with both arms (simultaneously or independently), or conducted with a single arm. The image capture device records and transmits these movements of the subject to the CPU, which becomes the first set of motion data. Additionally, the external body motion sensor(s) record and transmit motion data while the subject is performing this task, which becomes the second set of motion data. A trigger can be used to mark events into the recorded data. Specifically, an operator (and/or the patient) is asked to use the trigger to at the initiation of the task and asked to use the trigger at the completion of the task. In that manner, the trigger marks into the recorded data the start and end times for the task, which can be used to calculate the total duration of the experiment. The onset and offset of the trigger are calculated automatically by the CPU (Onset: first value greater than 0; Offset, last value greater than 0, for example on a trigger data channel). Onset, offset and total duration are displayed to the user, who can edit them if needed. Alternatively, in certain embodiments the trigger data could be automatically obtained from the motion data.

For this task, the image capture device records and transmits wrist joint position data (X, Y, Z) related to the 10 flexion-extension movements (ideally 20 movements total, 10 flexion and 10 extension movements). Those movements are filtered with a low-pass filter (cut-off 10 Hz, 11 coefficients) designed with the frequency sampling-based finite impulse response (FIR) filter design method. Then filtered X, Y, Z components are differentiated with a central difference algorithm to obtain velocities Vx, Vy, and Vz. Then, speed profiles are calculated as v=sqrt(Vx.\hat{ }2+Vy.\hat{ }2+Vz.\hat{ }2). Speed profiles are finally segmented (onset and offset are identified) to extract the 20 movements. To determine onset and offset of each of the 20 movements, X, Y, and Z are filtered with a FIR low-pass filter (cut-off 0.01 Hz, 11 coefficients); then velocities 1_Vx, 1_Vy, and 1_Vz are calculated by differentiating the filtered X, Y, and Z respectively, and speed profiles are calculated as v_1=sqrt(1_Vx.\hat{ }2+1_Vy.\hat{ }2+1_Vz.\hat{°}2). Peaks of (−v_1) are extracted automatically. This step identifies minimum values of v_1, which are assumed to be the same as minimum values of v (and easier to extract as the signal is less noisy). These points are used to define onset and offset of the single 20 movements. In this analysis and/or other embodiments of segmentation process, other methods can be used for extracting onset and offset values. For example a method based on thresholding speed or velocity profiles or a method based on zero crossings of position data or velocity components or a combination of the above, etc. could be used. Results of segmentation are displayed to the user who can edit them if needed. Segmentation of the movements can be confirmed by the data from the external body sensor. Ideally, both information from the image capture and external body sensor components is used together for the segmentation process (see below). For this task, at least one accelerometer (and or gyroscope) can also be mounted on the subject's index finger, wrist, or comparable joint location (e.g., a joint location which correlates with the movement being performed). During this task, the accelerometer data is processed with a 4^{th} order low-pass Butterworth filter with a cut-off frequency of 5 Hz. In other embodiments of analysis other types of filters designed with any method known in the art can be used, such as for example Window-based FIR filter design, Parks-McClellan optimal FIR filter design, infinite impulse response (IIR) filter, Butterworth filter, Savitzky-Golay filter, etc. Additionally, filters with different parameters and characteristics (coefficients/order, cut-off frequency, bandwidth, impulse response, step response, etc.) can be used. Analog filters and/or analog methods may be used where appropriate. Similarly differentiation can be performed using different algorithms, such as forward differencing, backward differencing, etc.

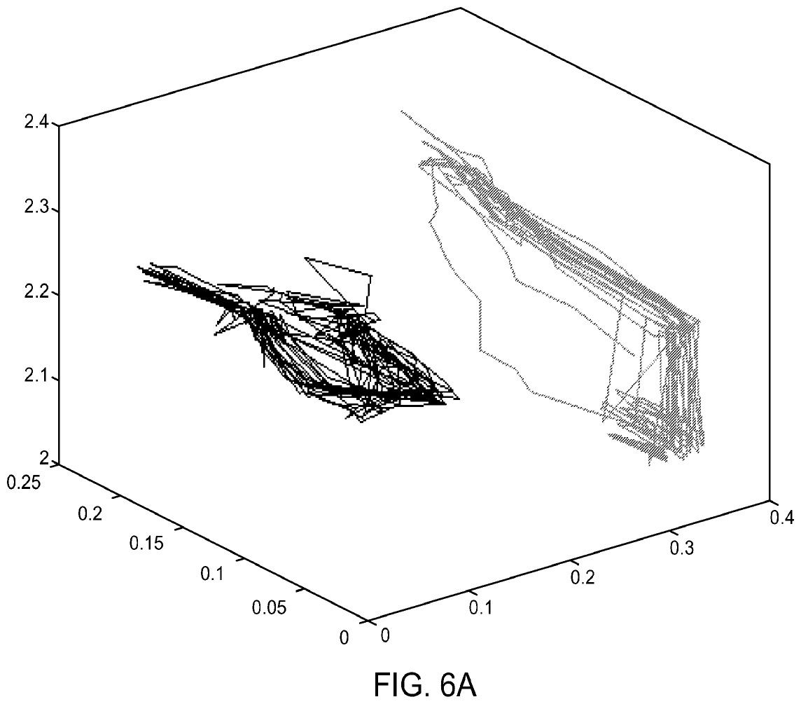

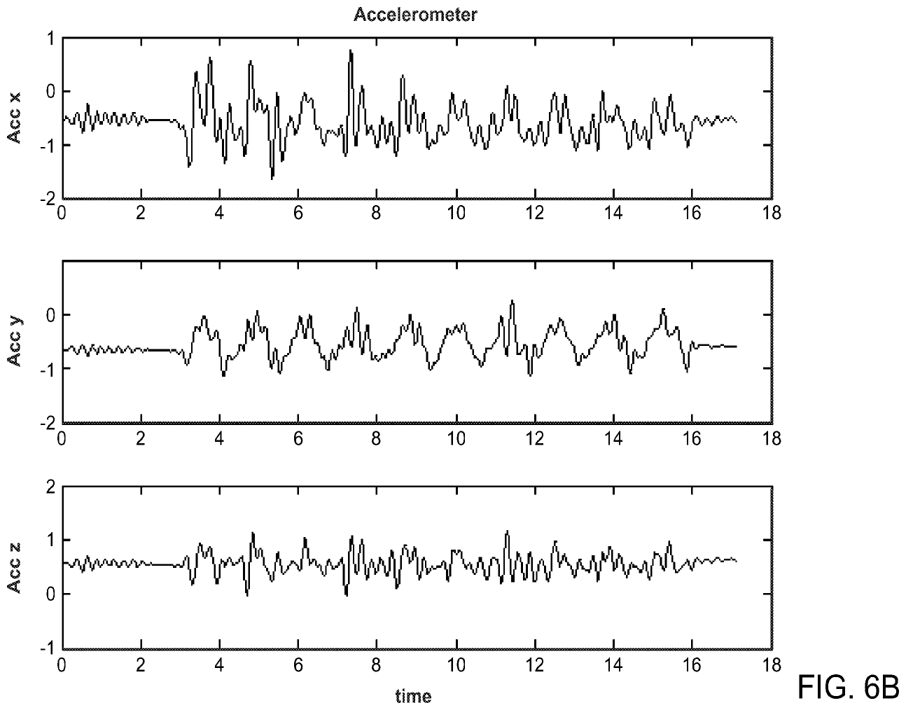

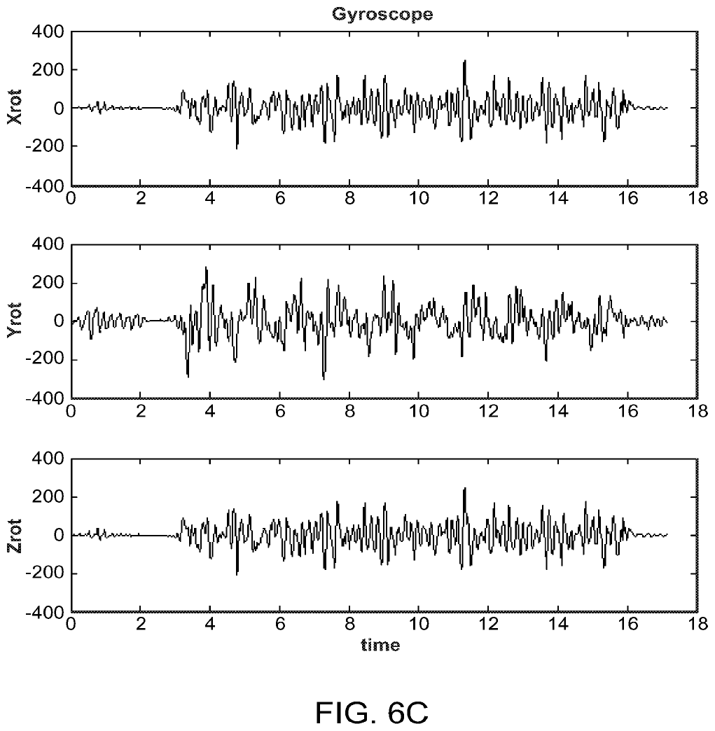

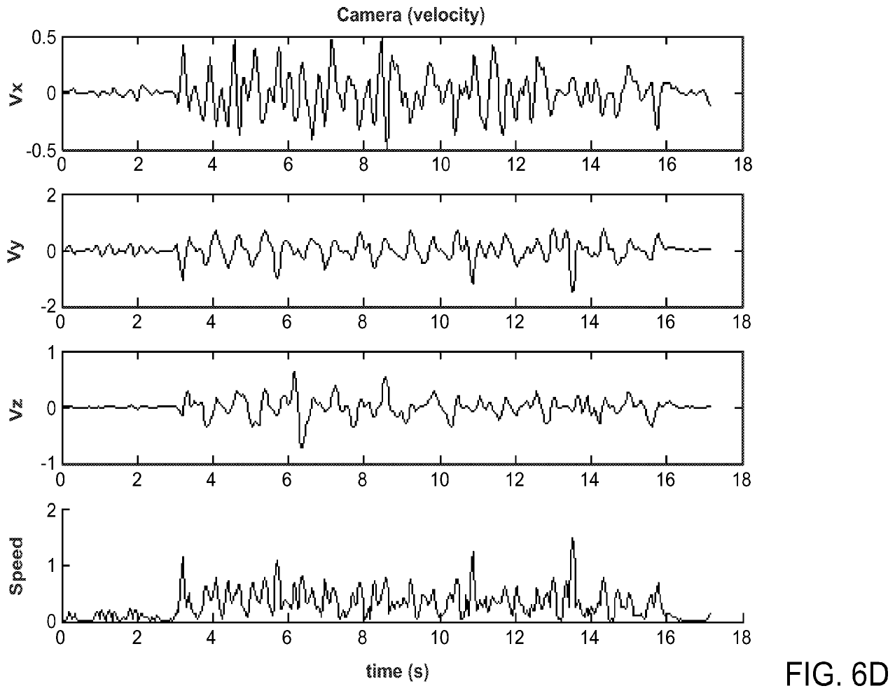

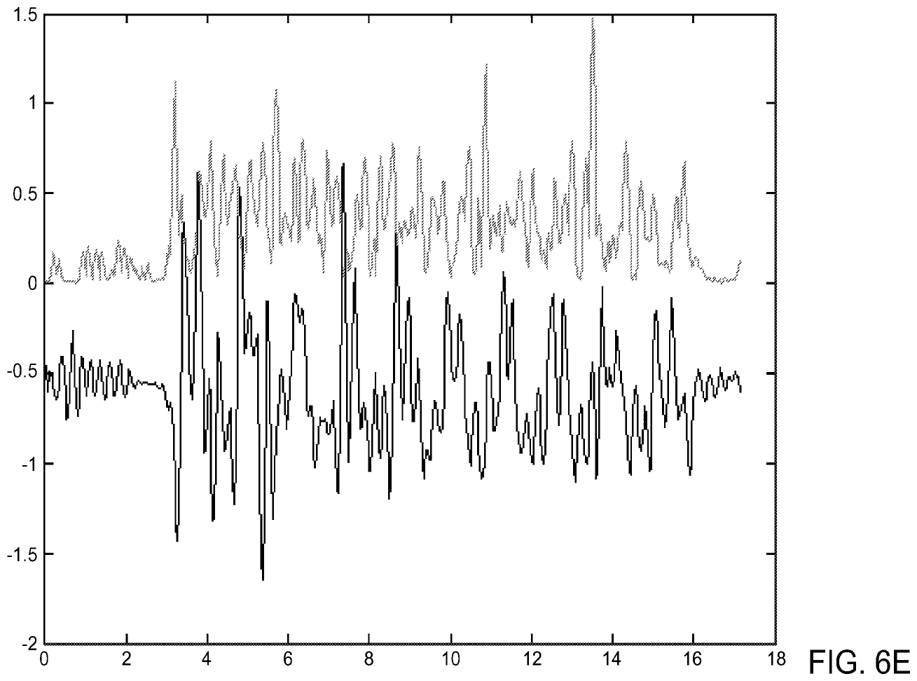

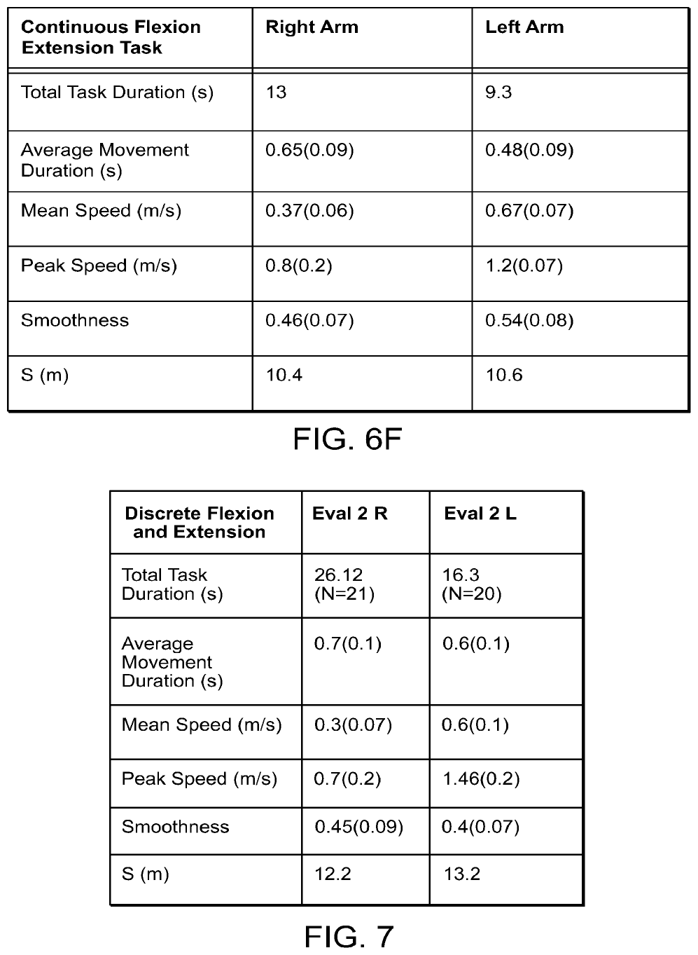

An example of this data and analysis for this task is shown in FIGS. 6A-6E. In FIG. 6A, a FIG. of position data recorded from the camera device indicating the position of the wrist in space, provided in X,Y,Z coordinates in the space of the subject, in the units of meters, during a test is provided. The blue line corresponds to the right wrist and the red line to the left wrist-note for tasks can be performed separately or together (this example data is for when the tasks for the left and right arm were performed individually, but demonstrated here on the same graph). In FIG. 6B, we provide the information from the accelerometers, provided in the X,Y,Z coordinates in the space relative to the accelerometer (i.e., relative to the measurement device) in relative units of the accelerometer—this data is for the right wrist. In FIG. 6C, we provide the information from the gyroscope in relative units of the gyroscope—this data is for the right wrist. In FIG. 6D, we provide the information of the velocity of movement in provided in X,Y,Z coordinates in the space of the subject, with the units of m/s, calculated based on the camera data—this data is for the right wrist. In FIG. 6E, we provide the information of the velocity (red line) based on the camera information in line with the data simultaneously recorded with the accelerometer (blue line)—note we provide the information in terms of m/s for the red line, and the blue line is given in terms of the relative units of the accelerometer—this data is for the right wrist (note the displayed Y-axis scale is in velocity and the x-axis is in units of time and the axis for the accelerometer is not shown as it is given in relative units). Here the accelerometer data was used to confirm the movements recorded with the camera (and vice versa), such that where one data set can be used to validate the other. The two sets of data can further be used such that the X,Y,Z acceleration information that was developed in the coordinate system of the accelerometer could be correlated to the X, Y, Z space of the patient's coordinate system.

The following metrics are extracted from the 20 segments of v, whose onset and offset is described above: movement mean speed (mean value of speed), movement peak speed (peak value of speed), movement duration: difference between offset of movement and onset of movement, movement smoothness (smoothness is a measure of movement quality that can be calculated as mean speed/peak speed; in this analysis and/or other embodiments smoothness can also be calculated as the number of speed peaks, the proportion of time that movement speed exceeds a given percentage of peak speed, the ratio of the area under the speed curve to the area under a similarly scaled, single-peaked speed profile, etc. Smoothness can also describe a general movement quality). Also calculated is the path length of the trajectory of the wrist joint (distance traveled in 3D space). See FIG. 6F.

The following metrics are the final output (kinematic and/or kinetic information) for this test: total duration of test; number of movements performed; movement mean speed (mean and standard deviation across all movements); movement peak speed (mean and standard deviation across all movements; movement duration (mean and standard deviation across all movements); movement smoothness (mean and standard deviation across all movements); and path length. In this analysis and/or other embodiments of the final output other statistical measures can be used such as for example variance, skewness, kurtosis, and/or high-order-moments. That data is then compared against the reference set or against the subject's prior set of kinematic and/or kinetic information for assessment of a movement disorder or assessment of the effectiveness of stimulation on treating the movement disorder.

In other embodiments, different computational methods and/or computational step order can be followed to determine kinematic and/or kinetic information output from the system. In certain embodiments, additional results that could be assessed include: acceleration (velocity, position, power, and/or other derived metrics (e.g., jerk)) as a function of joint(s) analyzed; acceleration (velocity, position, power, and/or other derived metrics (such as average, median, and/or standard deviation of these metrics)) as a function of movement(s) analyzed; acceleration (velocity, position, power, and/or other derived metrics) as a function of joint(s) position(s) analyzed; trajectory information (direction, quality, and/or other derived metrics) as a function of joint(s), movement(s), and/or joint(s) position(s); timing data related to movements (e.g., time to fatigue, time to change in frequency of power, time of task, time component of a task, time in position); joint or group joint data (absolution position, relative position, dimensions, velocity, position, power, time in position, and/or other derived metrics); and/or analysis based on individual or combined elements of these examples.

While in the above example, we provided a method where the relative accelerometer data was integrated with the camera image data to aid in the segmentation of movement data captured by the camera (see example FIG. 6) and to confirm the task movement was completed as directed, the two components' information can be integrated to provide further correlated information about movement that would not be captured by either device independently. For example, in a certain embodiment the power frequency spectrum of acceleration during movement of a specific joint as recorded by the accelerometer can be analyzed as a function of the movement recorded with the image device (or vice versa). As another example, in a certain embodiment the information from the camera position information can be used to determine constants of integration in assessing the information derived from the accelerometer which require an integration step(s) (e.g., velocity). As another example, an accelerometer on its own provides acceleration data relative to its own body (i.e., not in the same fixed coordinate system of a subject being analyzed with the system), and a camera cannot always provide all information about a joints information during complicated movements due its field of view being obscured by a subject performing complicated movement tasks, and by bringing the data together from the two components of the system the loss of information from either can be filled-in by the information provided by the correlated information between the two components. As another example, in a certain embodiment the camera image recordings can be used to correct drift in motion sensors (such as drift in an accelerometer or gyroscope). As another example, in a certain embodiment the camera image recordings can be used to register the placement and movement of the accelerometer (or other motion analysis sensor) in a fixed coordinate system (accelerometers X, Y, and Z recording/evaluation axes move with the device). In another example, in a certain embodiment the accelerometer (or other motion analysis sensor) can be placed on body locations that can be obscured from the recording view of the camera system during body movements. In another example, in a certain embodiment the camera information can be used to remove the effects of gravity on the accelerometer recordings (by being able to determine relative joint and accelerometer position during movement, and thus the relative accelerometer axis to a true coordinate space the subject is in and thus localize the direction of gravity). In another example, in a certain embodiment the acceleration data from the accelerometer (such as for example total acceleration) could be correlated and analyzed as a function of specific characteristics in movement determined from the camera component (such as for example an individual and/or a group of joints' position, movement direction, velocity). In another example, in a certain embodiment gyroscopic data and accelerometer data can be transformed into data in a patient's fixed reference frame by co-registering the data with the video image data captured by the camera and used to correct for drift in the motion sensors while simultaneously and/or allowing for the determination of information not captured by the camera system, such as for example when a patients' movements obscure a complete view of the patient and joints from the camera. In certain situations, a camera alone could suffer from certain disadvantages (for example an occlusion of views, software complexity for certain joints (e.g., hand and individual fingers), sensitivity to lighting conditions) but these advantages can be overcome by coupling the system with a motion sensors); while motion sensors (such as accelerometers and/or gyroscopes) alone suffer from certain disadvantages (for example drift and a lack of a fixed coordinate system) which can be overcome by coupling the system with with camera, for tasks and analysis that are important to the diagnosis, assessment, and following of movement disorders. In addition to these examples, more examples and alternative embodiments of the system follow describing the use of multiple motion analysis sensors (such as accelerometers, gyroscopes, and/or force plates), camera components, and/or other patient sensors (voice sensors, eye sensors, etc.) in various groups and combinations for the diagnosis, assessment, and following of movement disorders. These examples are only exemplary and not limiting, and the skilled artisan will appreciate that other methods of combining multiple components not mentioned here may be used with systems of the invention and that the components and task will be chosen to allow for assessment and/or diagnosis of the movement disorder being studied.

In another embodiment for assessing bradykinesia, a subject is asked to perform 10 arm flexion-extension movements (note that this number, joint, motion sensor(s) placement(s), and joint task is just exemplary). After each flexion or extension movement, the subject is asked to stop. The movements are performed as fast as possible. This test can then be repeated with both arms. The image capture device records and transmits these movements of the subject to the CPU, which becomes the first set of motion data. Additionally, the external body motion sensor(s) record and transmit motion data while the subject is performing this task, which becomes the second set of motion data. A trigger can be used to mark events into the recorded data. Specifically, an operator (and/or the patient) is asked to use the trigger at the initiation of the task and asked to use the trigger at the completion of the task. In that manner, the trigger marks into the recorded data the start and end times for the task, which can be used to calculate the total duration of the experiment. In certain embodiments, the trigger could mark a single event, a part of an event, and/or multiple events or parts of event, such as all 10 flexion movements. The onset and offset of the trigger are calculated automatically by the CPU (Onset: first value greater than 0; Offest, last value greater than 0). Onset, offset and total duration are displayed to the user, who can edit them if needed. Alternatively, in certain embodiments the trigger data could be automatically obtained from the motion data.

For this task, the image capture device records and transmits wrist joint position data (X, Y, Z) related to the 10 flexion-extension movements (ideally 20 movements total 10 flexion and 10 extension movements). Similarly, the accelerometer and optionally gyroscope are positioned on the wrist joint.

The data are analyzed similarly to above, but segmentation of speed profiles is performed differently such that the accelerometer (+gyroscope) data are scaled to be same length as the image capture data and the process of segmentation to extract the 20 single movements uses gyroscope data. Specifically the Z component of the data recorded from the gyroscope is analyzed to extract peaks; starting at the time instant corresponding to each identified peak, the recording is scanned backward (left) and forward (right) to find the time instants where the Z component reaches 5% of the peak value (note in alternative embodiments other thresholds could be used (For example, such as 2%, 3%, 4%, 10%, 15% of peak value, depending on the signal-to-noise ratio.)). The time instants at the left and right are identified respectively as the onset and offset of the single movement (corresponding to the identified peak). This segmentation process leads to extraction of 10 movements. A similar process is repeated for the -Z component of the data recorded from the gyroscope to identify the remaining 10 movements.

The following metrics are extracted from the 20 segments of v, whose onset and offset is described above: movement mean speed (mean value of speed), movement peak speed (peak value of speed), movement duration (difference between offset of movement and onset of movement), movement smoothness (mean speed/peak speed). Also calculated is the path length of the trajectory of the wrist joint (distance traveled in 3D space). Following a process similar to above, detailed in FIG. 6A-E, the data in FIG. 7, was determined.

The following metrics are the final output (kinematic and/or kinetic information) for this test: total duration of test; number of movements performed; movement mean speed (mean and standard deviation across all movements); movement peak speed (mean and standard deviation across all movements; movement duration (mean and standard deviation across all movements); movement smoothness (mean and standard deviation across all movements); and path length.

That data is then compared against the reference set or against the subject's prior set of kinematic and/or kinetic information for assessment of a movement disorder or assessment of the effectiveness of stimulation on treating the movement disorder. As above, in other embodiments, different computational methods and/or step order can be followed to determine kinematic and/or kinetic information output from the system.

In still another embodiment, a subject is asked to perform 10 hand opening and closing movements, as fast as possible, while the hand is positioned at a fixed location (here for example the shoulder)—note that this number, joint, motion sensor(s) placement(s), and joint task is just exemplary. The image capture device records and transmits these movements of the subject to the CPU, which becomes the first set of motion data. This camera data can be used to assess if the patient is keeping their hand in a fixed location, for example by analyzing wrist or arm positions. Or in alternative embodiments, the camera data can be used to determine individual characteristics of the hand motion (such as for example individual finger positions) when assessed in conjunction with the accelerometer. Additionally, the external body motion sensor(s) record and transmit motion data while the subject is performing this task, which becomes the second set of motion data. A trigger can be used to mark events into the recorded data. Specifically, an operator (and/or the patient) is asked to use the trigger to at the initiation of the task and asked to use the trigger at the completion of the task (herein the last evaluated hand open-closing task). In that manner, the trigger marks into the recorded data the start and end times for the task, which can be used to calculate the total duration of the experiment. The onset and offset of the trigger are calculated automatically by the CPU (Onset: first value greater than 0; Offest, last value greater than 0). Onset, offset and total duration are displayed to the user, who can edit them if needed.

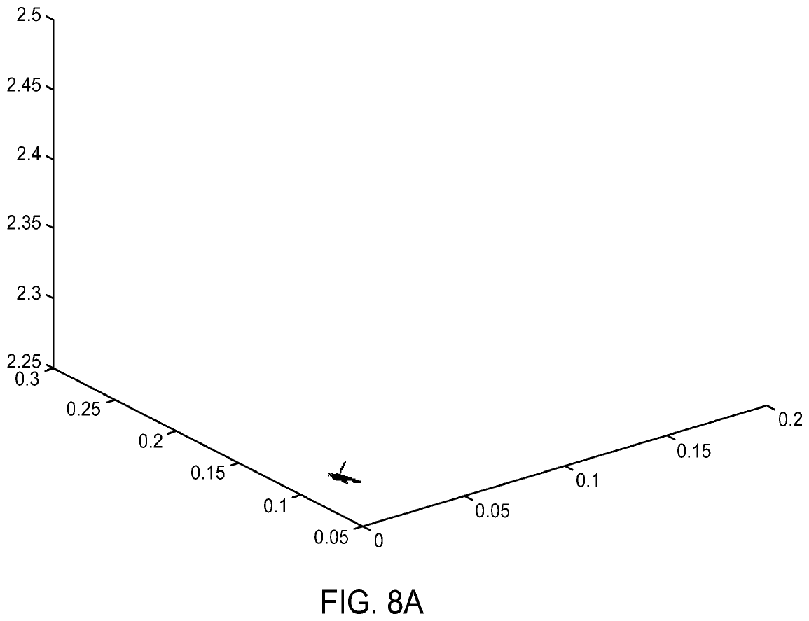

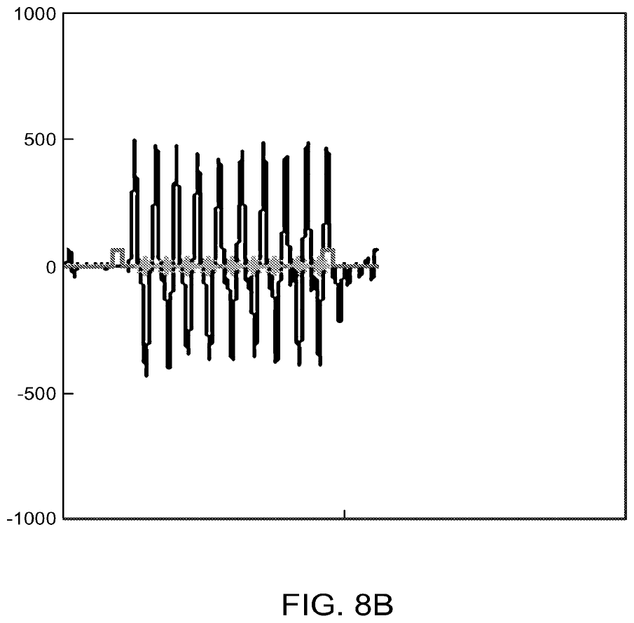

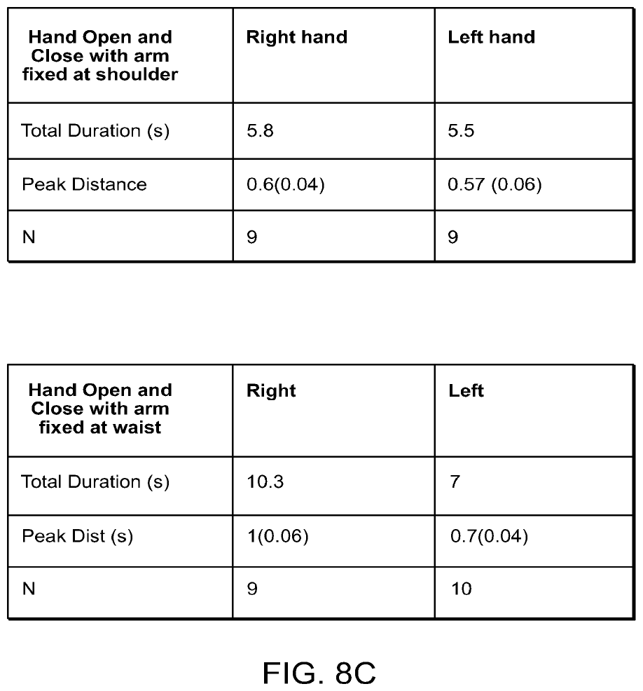

For this task, the image capture device records and transmits wrist joint position data (X, Y, Z) in a fixed coordinate space related to the opening and closing of the hand. Or alternatively, the image capture device is used to validate the position of the wrist and arm, and thus that the hand is fixed at the location chosen for the movement task evaluation (for example here at the shoulder), see FIG. 8A depicting wrist position. In FIG. 8A, the position of the hand is gathered from the data, as can be noticed compared to FIG. 6A the patient was able to follow the instructions of keeping the hand stable, as the limited movement was determined within the normal range in the case of the patient (e.g., the patient did not demonstrate the same range of movement depicted in the flexion and extension movement), and at point in X, Y, Z space of the patient that corresponds to the appropriate anatomical level (e.g., shoulder). In alternative tasks, the relative hand position can be tracked with a camera, and be used to determine what effect the location of the hand has on the hand open and closing speeds as determined with accelerometer and/or gyroscope data (see below). With a single camera alone it would not be possible to study all aspects of individual fingers as the hand closes (such as for example due to occlusion of views), yet accelerometer and gyroscopic data can fill this void; furthermore, the gyroscope and accelerometer cannot provide fixed joint position information (as the observation axes are dependent on the position of the recording systems); the combined information is particularly important for the diagnosis, evaluation, and following of movement disorders.

Similarly, the accelerometer and optionally gyroscope are positioned on the subject's index finger. Gyroscopic and acceleration data of the index finger is recorded. For example, in FIG. 8B, peaks of the rotational component of the gyroscope along its X axis is identified and displayed to the user (blue line in units of the gyroscopic device), the red lines show the triggering device, and the green line demonstrates the peak locations of the movements. The gyroscopic information, corresponding to the waveform characteristics of the data could be used to determine the time point when the hand was opened or closed (based on the rotational velocity approaching zero at this point). The distance between consecutive peaks (a measure of the time between two consecutive hand closing/opening movements) is calculated. The number of movements performed is calculated as the number of peaks+1. See FIG. 8C (top half for data gathered with the hand held at the shoulder). In FIG. 8C (bottom half), this same data is provided for the hand held at the waist, as confirmed by the camera system in a fixed coordinate space. The difference in hand speeds in these positions can only be confirmed through the use of data from both the image capture device and the external body sensors.

The following metrics are the final output for this test: total duration of test; number of movements performed; and time between two consecutive hand closing/opening movements (mean and standard deviation across all movements). That data is then compared against the reference set or against the subject's prior set of kinematic and/or kinetic information for assessment of a movement disorder or assessment of the effectiveness of stimulation on treating the movement disorder. As above, in other embodiments, different computational methods and/or step order can be followed to determine kinematic and/or kinetic information output from the system.

In still another embodiment, a subject is asked to perform combined movements (flexion followed by hand opening/closing followed by extension followed by hand opening/closing) 10 times as fast as possible (note that this number, joint, motion sensor(s) placement(s), and joint task is just exemplary).

The image capture device records and transmits these movements of the subject to the CPU, which becomes the first set of motion data. Additionally, the external body motion sensor(s) record and transmit motion data while the subject is performing this task, which becomes the second set of motion data. A trigger can be used to mark events into the recorded data. Specifically, an operator (and/or the patient) is asked to use the trigger at the initiation of the task and asked to use the trigger at the completion of the task. In that manner, the trigger marks into the recorded data the start and end times for the task, which can be used to calculate the total duration of the experiment. The onset and offset of the trigger are calculated automatically by the CPU (Onset: first value greater than 0; Offest, last value greater than 0). Onset, offset and total duration are displayed to the user, who can edit them if needed.

The final output is total duration of test, and a combination of the above data described in the individual tests. That data is then compared against the reference set or against the subject's prior set of kinematic and/or kinetic information for assessment of a movement disorder or assessment of the effectiveness of stimulation on treating the movement disorder. In alternative tasks, more complicated movements can be performed where the movements are occurring simultaneously. As above, in other embodiments, different computational methods and/or step order can be followed to determine kinematic and/or kinetic information output from the system.

In still another embodiment, a subject is asked to touch their nose with their index finger, as completely as possible, 5 times (note that this number, joint, motion sensor(s) placement(s), and joint task is just exemplary). The image capture device records and transmits these movements of the subject to the CPU, which becomes the first set of motion data. Additionally, the external body motion sensor(s) record and transmit motion data while the subject is performing this task, which becomes the second set of motion data. A trigger can be used to mark events into the recorded data. Specifically, an operator (and/or the patient) is asked to use the trigger to at the initiation of the task and asked to use the trigger at the completion of the task. In that manner, the trigger marks into the recorded data the start and end times for the task, which can be used to calculate the total duration of the experiment. The onset and offset of the trigger are calculated automatically by the CPU (Onset: first value greater than 0; Offest, last value greater than 0). Onset, offset and total duration are displayed to the user, who can edit them if needed.

For this task, the image capture device records and transmits wrist joint position data (X, Y, Z). The accelerometer and optionally gyroscope are positioned on the subject's index finger.

For this task, the image capture device records and transmits wrist joint position data (X, Y, Z). Those movements are filtered with a FIR low-pass filter (cut-off 10 Hz, 11 coefficients). Then filtered X, Y, Z are differentiated to obtain velocities Vx, Vy, and Vz. Then, speed profiles are calculated as v=sqrt(Vx.\hat{ }2+Vy.\hat{ }2+Vz.\hat{ }2). Speed profiles are finally segmented (onset and offset are identified) to extract the 20 movements. To determine onset and offset of each of the 20 movements, X, Y, and Z are filtered with a FIR low-pass filter (cut-off 0.01 Hz, 10 coefficients); then velocities 1_Vx, 1_Vy, and 1_Vz are calculated by differentiating the filtered X, Y, and Z respectively, and speed profiles are calculated as v_1=sqrt(1_Vx.\hat{ }2+1_Vy.\hat{ }2+1_Vz.\hat{ }2). Peaks of (−v_1) are extracted automatically. This step identifies minimum values of v_1, which are assumed to be the same as minimum values of v (but easier to extract as the signal is less noisy). These points are used to define onset and offset of the single 20 movements. Results of segmentation are displayed to the user who can edit them if needed.

Acceleration is calculated as root square of Acc_X, Acc_Y, and Acc_Z, which are recorded with the accelerometer. Signal is de-trended (mean is removed), FFT magnitude is calculated (N=1024), and values between 6 and 9 Hz (as well as between 6 and 11 Hz) are summed. The resulting value represents the power of the signal in the range 6-9 Hz (or 6-11 Hz). For this example, tremor is calculated as the power of the signal in the range 6-9 Hz (or 6-11 Hz) divided by the total power of the signal.

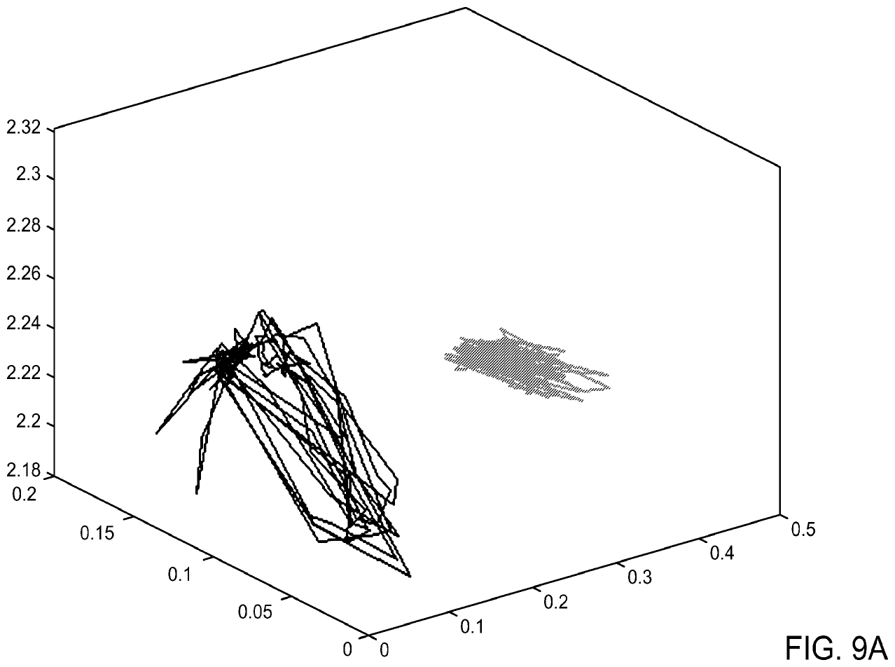

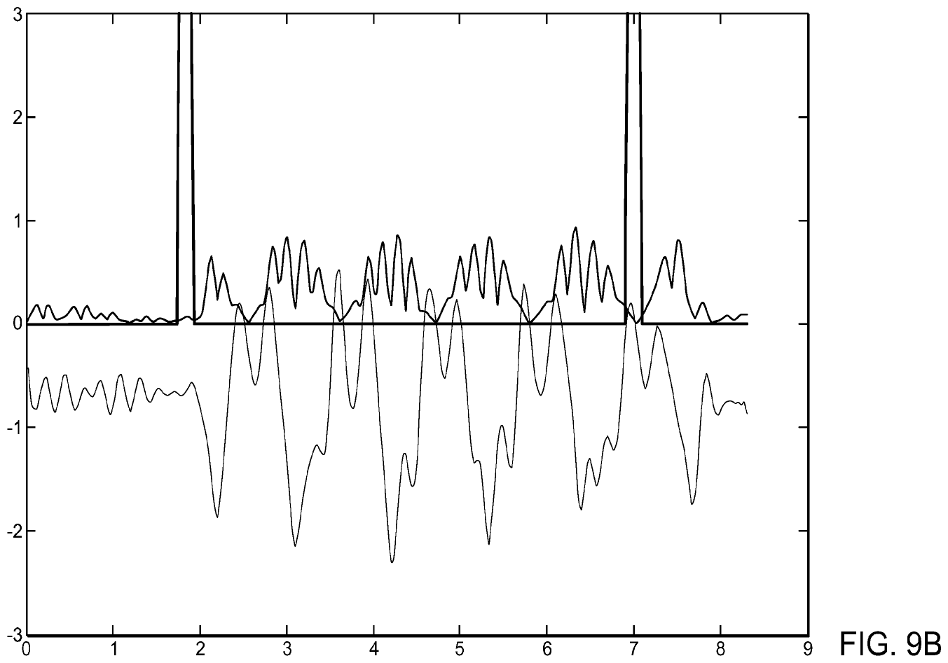

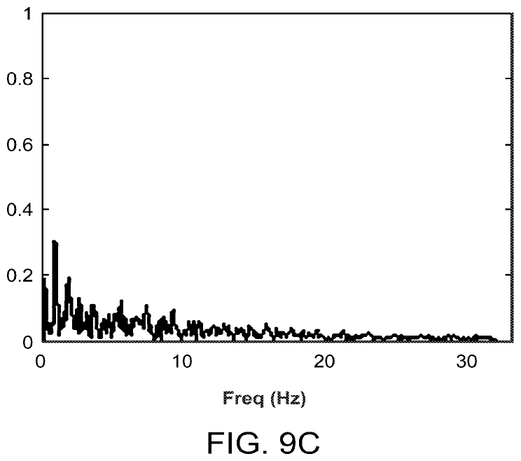

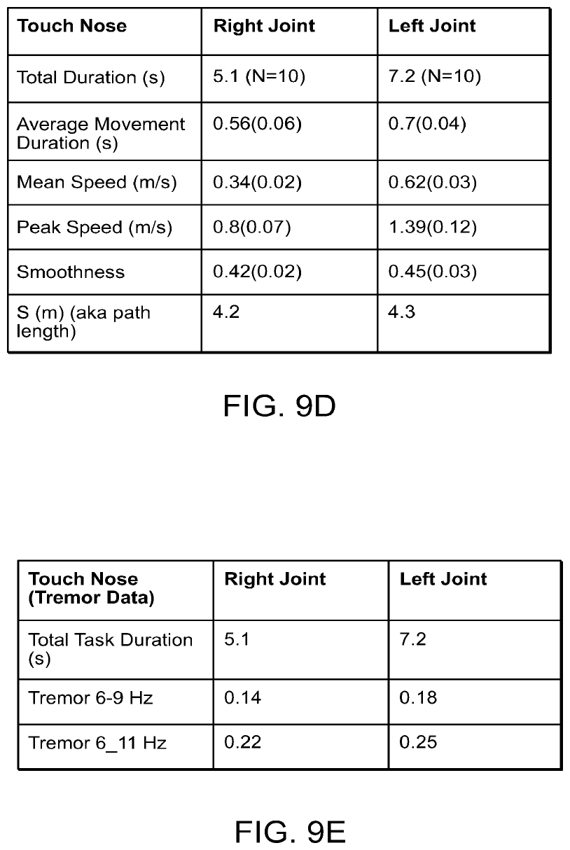

In FIG. 9A, we show an example of position data recorded by the camera provided in X,Y,Z coordinates in the space of the subject, in the units of meters, during the test. The blue line corresponds to the right wrist and the red line to the left wrist—note for tasks can be performed separately or together (this example data is for when the tasks for the left and right arm were performed individually). In FIG. 9B, we show velocity determined from the camera data (red), accelerometer data (blue line), and the trigger data to mark the beginning of the first and last movement (black lines)—the y axis is given in m/s for the velocity data (note the accelerometer data is provided in relative units of the accelerometer) and x-axis is time, this data is for the right joint. In FIG. 9C, we show the data from the power in the movements of the right hand as a function of frequency as determined from the accelerometer data.

The following metrics are the final output for this test: total duration of test; number of movements actually performed; movement mean speed (mean and standard deviation across all movements); movement peak speed (mean and standard deviation across all movements); movement duration (mean and standard deviation across all movements); movement smoothness (mean and standard deviation across all movements); path length; tremor in the range 6-9 Hz; tremor in the range 6-11 Hz. See FIG. 9D and FIG. 9E. In other embodiments this analysis could be done in other bands, such as for example from 8 to 12 Hz or at one specific frequency. That data is then compared against the reference set or against the subject's prior set of kinematic and/or kinetic information for assessment of a movement disorder or assessment of the effectiveness of stimulation on treating the movement disorder. As above, in other embodiments, different computational methods and/or step order can be followed to determine kinematic and/or kinetic information output from the system.

In an additional example, we can further determine further information about the movement characteristics with the data recorded from the camera and the accelerometer. For example, the tremor data could be analyzed for each individual movement and correlated to the camera information to provide true directional information (i.e., the tremor as a function of movement direction or movement type) or quality of movement information. An individual system of just the accelerometer could not provide such information, because the accelerometer reports its acceleration information as a function of the internal axes of the accelerometer that are changing continuously with the patient movement. Furthermore, typical camera systems cannot provide this information because their sampling rate is generally too low (for example see similar tremor data gathered with a typical camera during the same movements), nor do they allow one to localize tremor to a specific fixed location on the body with a single fixed camera as patient movements can obscure joint locations from observation (i.e., a single camera could not provide full 3D information about the movements, and multiple cameras still cannot fill information when their views are obscured by patient movements). In certain examples, a high speed camera could be used to provide tremor data (and/or with the use of other motion analysis systems) Furthermore, in certain embodiments the combined system allows multiple levels of redundancy that allow for a more robust data set that can provide further details and resolution to the signal analysis of the patient data.

In another embodiment, resting tremor is assessed, which is assessment of tremor while the hand is at a resting position (for example evaluated from 4-6 Hz). In another embodiment, postural tremor is assessed while having a subject maintain a fixed posture with a joint. For example, a subject is asked to keep their hand still and hold it in front of their face. During the assessments for resting, action, and/or postural tremor, different frequency bands can be explored, such as frequencies or frequency bands from 0-1 Hz, 1-2 Hz, 2-3 Hz, 4-6 Hz, 8-12 Hz, and so on. The tremor frequency band could be determined based on a specific disease state, such as Essential Tremor and/or Parkinson/s Disease (or used to compare disease states).

In another embodiment, a patient's posture and/or balance characteristics are assessed. A subject is asked to stand on a force plate (e.g., a Wii Balance Board) while multiple conditions are assessed: eyes open, eyes closed, patient response to an external stimuli (e.g., an clinical evaluator provides a push or pull to slightly off balance the patient, or a mechanical system or robotic system provides a fixed perturbation force to the patient) herein referred to as sway tests (note that this set of conditions is just exemplary, and that other conditions could be completed, or just a subset of those presented. Furthermore, in certain embodiments the tests could be completed with more or less body motion sensors placed at different locations on the body, and/or the analysis can be completed for different joint(s)). During measurements with eyes open or closed, the subject is simply asked to stand on a force plate. During sway measurements, the subject is slightly pulled by a clinician (or other system, such as a mechanical or robotic system). The image capture device can optionally record and transmit these movements of the subject to the CPU, which becomes the first set of motion data. Additionally, the external body motion sensor(s) record and transmit motion data while the subject is performing this task, which becomes the second set of motion data. Data from the force plate is also acquired and transmitted to the CPU, which becomes the balance data. A trigger can be used to mark events into the recorded data. Specifically, an operator (and/or the patient) is asked to use the trigger to at the initiation of the task and asked to use the trigger at the completion of the task. In that manner, the trigger marks into the recorded data the start and end times for the task, which can be used to calculate the total duration of the experiment. The onset and offset of the trigger are calculated automatically by the CPU (Onset: first value greater than 0; Offset is Onset+15 seconds (or Onset+total length of data if recordings are shorter)). Onset, offset and total duration are displayed to the user, who can edit them if needed.

For this task, the image capture device can record and transmits joint position data (X, Y, Z) related to patient spinal, shoulder, and/or additional joint information. The accelerometer and optionally gyroscope are positioned on the subject's spinal L5 location (on the surface of the lower back) and/or other joint locations.

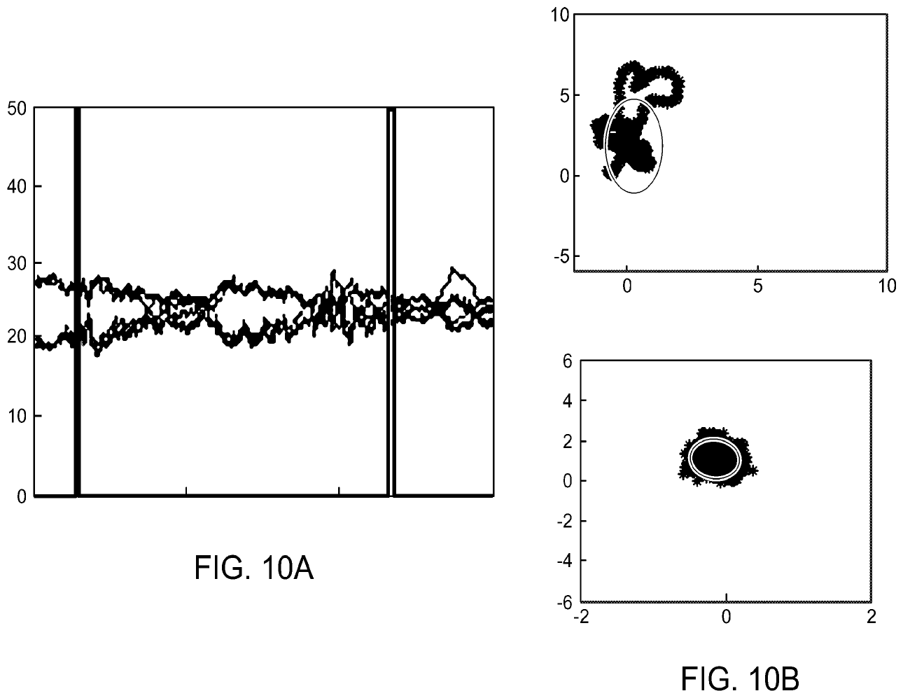

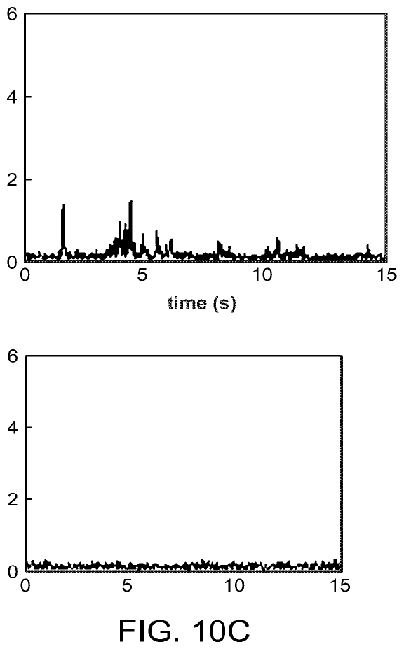

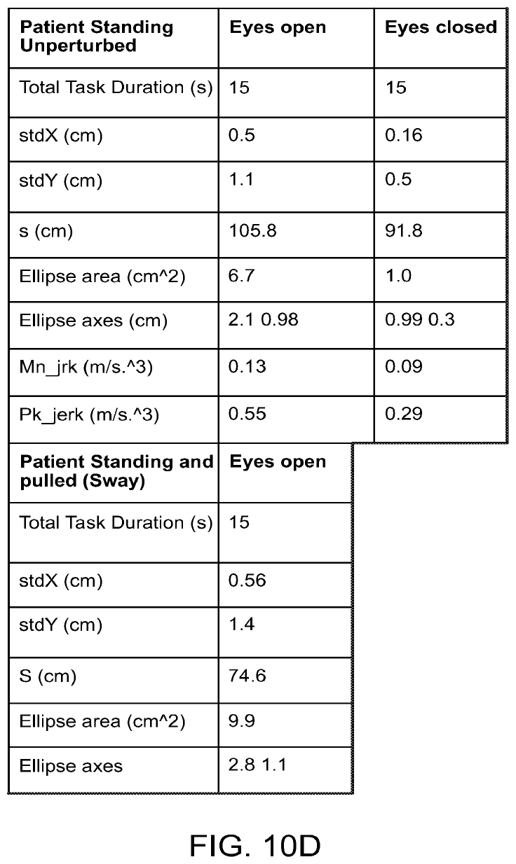

Metrics of balance are derived from the center of pressure (X and Y coordinates) recordings of the force plate. StdX and StdY are calculated as the standard deviation of the center of pressure. The path length of the center of pressure (distance traveled by the center of pressure in the X, Y plane) is also calculated. The movements of the center of pressure are fitted with an ellipse, and the area and axes of the ellipse are calculated. The axes of the ellipse are calculated from the eigenvalues of the covariance matrix; the area is the product of the axes multiplied by PI. In FIG. 10A, the weight calculated for the front and back of the left and right foot is calculated in kg, and the red line depicts a trigger mark where a clinician has determined a patient has stepped on the board and begun balancing and the second line depicts when the clinician tells the patient the test is over and they can prepare to get off the force plate, the x-axis is in until of time. In FIG. 10B, we show typical examples of data depicting patients center of gravity movements (blue), here depicted in units of length, and area ellipses depicting total movement (red)—the top part shows a patient who has been perturbed (eyes open) and swaying and the bottom part shows a patient standing without perturbation (eyes closed). The time information could be communicated on a third axis or via color coding, here for clarity it is removed in the current depiction,

Jerk is calculated by analyzing acceleration along X and Y (and Z in certain embodiments), which are calculated by differentiating accelerometer data along the relevant axes, smoothed with a moving average filter (N=5). Jerk is then calculated as root square of Jerk X and Jerk Y (and in certain embodiments as the root square of Jerk X, Jerk Y, and Jerk Z). Mean value and peak value of jerk is calculated. In FIG. 10C the jerk data, in units of position per time cubed, are provided—the top part shows a patient who has been perturbed and swaying (eyes open) and the bottom part shows a patient standing without perturbation (eyes closed)—corresponding to the data in FIG. 10B. The jerk data can be calculated in the X and Y axis from the force plate, and X, Y, and Z dimensions from the accelerometer data or image capture device data (note each captures different jerk information, for example from the force plate we could calculate jerk of the center of gravity, from the accelerometers the jerk about the individual axes of the devices, and for the camera the relative jerk data of the analyzed joints. All of these measures can be compared and registered in the same analysis space by appropriately coupling or co-registering the data as mentioned above). The image capture device can capture information about the joint positions and be analyzed similar to what is described in the above examples.

In the sway condition, all of the metrics can be evaluated as a function of the initial subject perturbation, push or pull force, derived perturbation characteristics, and/or derived force characteristics (such as rate of change, integral of force, force as function of time, etc).

The following metrics are the final output for this test: total duration of test; StdX; StdY; path length s; ellipse area; ellipse major and minor axis; mean jerk; and peak jerk, see FIG. 10D. That data is then compared against the reference set or against the subject's prior set of kinematic and/or kinetic information for assessment of a movement disorder or assessment of the effectiveness of stimulation on treating the movement disorder. In certain embodiments, this method allows an observer to provide a controlled version of a typical Romberg test used in clinical neurology. As above, in other embodiments, different computational methods and/or step order can be followed to determine kinematic and/or kinetic information output from the system. In certain embodiments, additional results that could be assessed include: center of gravity (and/or its acceleration, velocity, position, power, and/or other derived metrics (e.g., jerk)) as a function of joint(s) analyzed; body position and/or joint angle (and/or their acceleration, velocity, position, power, and/or other derived metrics (such as average, median, and/or standard deviation of these metrics)) as a function of movement(s) analyzed; sway trajectory information (acceleration, velocity, position, power, direction, quality, and/or other derived metrics) as a function of patient perturbation force (acceleration, velocity, position, power, direction, quality, and/or other derived metrics); timing data related to the patients COG movement (e.g., time to return to center balanced point, time of sway in a certain direction(s)); and/or analysis based on individual or combined elements of these and/or the above examples.

In another embodiment for assessing gait and/or posture, a subject is asked to walk 10 meters, four different times (note that this number, joint, motion sensor(s) placement(s), and joint task is just exemplary). The image capture device optionally records and transmits these movements of the subject to the CPU, which becomes the first set of motion data. Additionally, the external body motion sensor(s) record and transmit motion data while the subject is performing this task, which becomes the second set of motion data. The trigger is used to mark events into the recorded data. Specifically, the subject is asked to use the trigger to at the initiation of the task and asked to use the trigger at the completion of the task. In that manner, the trigger marks into the recorded data the start and end times for the task, which can be used to calculate the total duration of the experiment. The onset and offset of the trigger are calculated automatically by the CPU (Onset: first value greater than 0; Offset, last value greater than 0). Onset, offset and total duration are displayed to the user, who can edit them if needed.

For walks 1 and 2, an external body motion sensor (accelerometer and gyroscope) is positioned on the subject's left and right ankles. For this task, the image capture device can record and transmits joint position data (X, Y, Z) related to joints of the lower limbs, spine, trunk, and/or upper limbs. For this task, the image capture device can record and transmits joint position data (X, Y, Z) related to joints of the lower limbs, spine, trunk, and/or upper limbs.

For walks 3 and 4, a first external body motion sensor (accelerometer and gyroscope) is positioned on the subject's back (L5), and a second external body motion sensor (accelerometer and gyroscope) is positioned on one of the subject's ankles, preferably the right ankle. For this task, the image capture device can record and transmits joint position data (X, Y, Z) related to joints of the lower limbs, spine, trunk, and/or upper limbs.

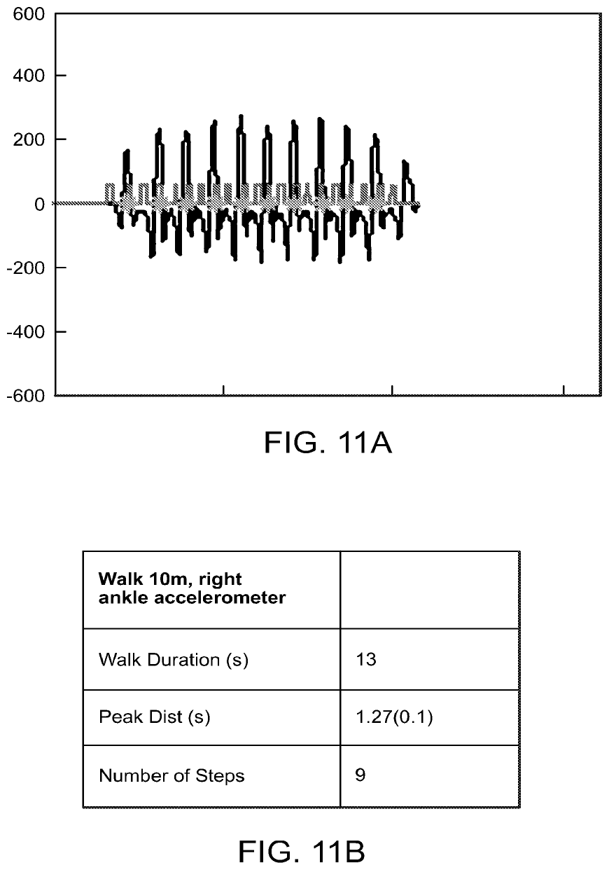

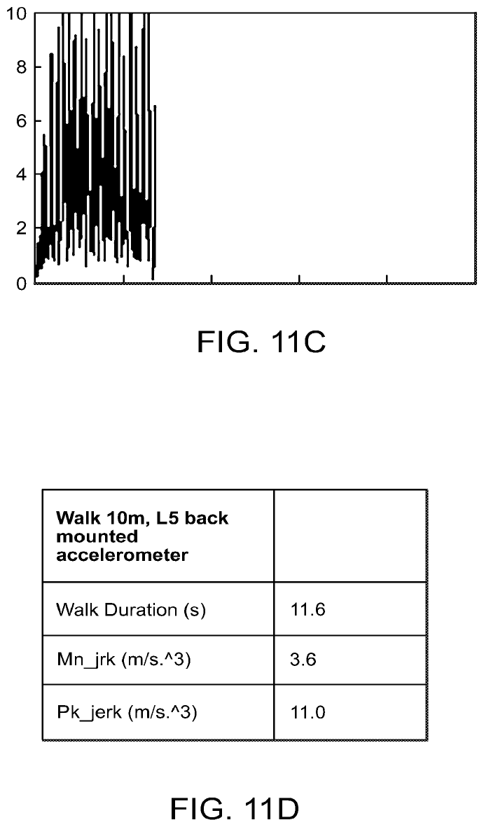

For the right ankle, acceleration metrics of gait are derived. Specifically peaks of Z rot (gyroscope data for Z) are extracted, and the distance in time between consecutive peaks is calculated (this is considered a metric of stride time). The number of strides is calculated as number of peaks+1. For example in FIG. 11A, the peaks of the rotational component of the gyroscope along its Z axis are identified and displayed to the user (blue line in units of the gyroscopic device), the red lines show the triggering device, and the green line depicts the time instants corresponding to peaks of Z rotational component. The Y-axis is given in the relative units of the gyroscope around its Z-axis, and the X-axis in units of time. The triggering device here is activated on every step. The compiled results of this analysis are shown in FIG. 11B, demonstrating the total walk time, and longest time per right step (Peak Distance).

For the left ankle, acceleration metrics of gait are derived as described above for the right ankle, but -Zrot is used instead.

For the back, acceleration is calculated by analyzing jerk along X, Y, and Z, which is calculated by differentiating accelerometer data along X, Y, and Z. Jerk_X, Jerk_Y and Jerk_Z are then filtered with a low-pass butterworth filter (N=4, cut_off=5 Hz). Jerk is finally calculated as root square of Jerk_X, Jerk_Y, and Jerk_Z. In FIG. 11C, an example of Jerk is shown (the Y-axis is in the units of m/time\hat{ }3, X-axis in terms of time), the blue line corresponds to the period while a person is walking and the open space when the walk and task recording has stopped. Mean value and peak value of jerk is calculated. The image capture device can capture information about the joint positions and be analyzed similar to what is described in the above examples. The compiled results of this analysis are shown in FIG. 11D.

The following metrics for walks 1 and 2 are the final output for this test: total duration of test (average of test 1 and test 2); mean stride time for left ankle (average of test 1 and test 2); standard deviation of stride time for left ankle (average of test 1 and test 2); number of strides for right ankle; mean stride time for right ankle (average of test 1 and test 2); standard deviation of stride time for right ankle (average of test 1 and test 2); and number of strides for right ankle. That data is then compared against the reference set or against the subject's prior set of kinematic and/or kinetic information for assessment of a movement disorder or assessment of the effectiveness of stimulation on treating the movement disorder.

The following metrics for walks 3 and 4 are the final output for this test: total duration of test; mean jerk (average of test 3 and test 4); and peak jerk (average of test 3 and test 4). That data is then compared against the reference set or against the subject's prior set of kinematic and/or kinetic information for assessment of a movement disorder or assessment of the effectiveness of stimulation on treating the movement disorder. As above, in other embodiments, different computational methods and/or step order can be followed to determine kinematic and/or kinetic information output from the system.

Many other metrics could be determined, such as for example average step length, path direction (e.g., was a patient moving in a straight line or some other path), time foot is planted, stride length, range of motion of joint, relative arm and leg movement characteristics, and posture during movements.

### Wearable Components

In alternative embodiments, the system components described herein, can in part or in whole be part of a wearable item(s) that integrates some or all of the components. For example a person could wear a suit that integrates motion analysis sensors (e.g., accelerometers) in a wearable item, with a CPU processing unit, a telecommunications component and/or a storage component to complete the analysis, diagnosis, evaluation, and/or following of a movement disorder. Or for example one could have a watch with an accelerometer connected wireles sly to a mobile phone and an external image capture device to complete the analysis, diagnosis, evaluation, and/or following of a movement disorder (in certain embodiments the image capture camera could be in a mobile phone, and/or part of a watch or wearable item). In certain embodiments the system can contain a wearable image capture device (such as for example components exemplified by a GoPro camera and/or image capture devices typically worn by the military or law enforcement). In certain embodiments, the wearable system components can be integrated (either wirelessly or via wired connections) with multiple other wearable components (such as a watch, a helmet, a brace on the lower limb, a glove, shoe, and/or a shirt). In certain embodiments, the patient could wear a shoe that has at least one sensor built into the system, such as for example a sole of a shoe that can measure the force or pressure exerted by the foot, such as for example a component that could be used to provide a pressure map of the foot, displays force vs. time graphs and pressure profiles in real time, and/or position and trajectories for Center of Force (CoF) during phases of gait.

In certain embodiments, the system can track and/or compare the results of two or more different users, for example two people could be wearing comparable wearable items, such that the items are part of the same network with at least one CPU unit, which allows the comparison of individual's wearing the devices (for example a healthy individual could be wearing a device that is simultaneously worn by another who was suffering from a movement disorder, and the individuals could preform tasks simultaneously, such that the CPU could compare the data from the wearable items to complete analysis, diagnosis, evaluation, and/or following of a movement disorder). In alternative embodiments, at least one of the wearable items can be connected or integrated with an active component(s) (such as a for example a robotic or electromechanical systems that can assist in controlled movements) so for example a healthy individual could be wearing a device that is simultaneously worn by another who was suffering from a movement disorder, and the individuals could preform tasks simultaneously, such that the CPU could compare the data from the wearable items and provide a signal that controls active components of the device worn by the individual suffering from a movement disorder to aid or assist the patient in the completion of a task (this for example could be used as part of a training or therapy protocol). In alternative embodiments, the systems could be connected via active and passive feedback mechanisms.

In yet a further alternative embodiment, multiple components and/or systems could integrated through the methods described herein and be used for the analysis of multiple individuals, such as for example following the performance of a sports team during tasks or competition.

In one alternative embodiment, one could use implantable components, where at least one motion analysis component is implanted in the body of a subject being analyzed. This embodiment requires invasive procedures to place a sensor in the body.

In certain embodiments, the system could be used as a training device with or without feedback, such as in training surgeons to preform movements for surgical procedures (such as without tremor or deviation from predefined criteria), or an athlete completing balance training.

### Active System Components

In alternative embodiments, the motion analysis system may be integrated with an active component(s) (such as a for example a robotic or electromechanical systems that can assist in controlled movements), which for example could assist the patient in movement tasks. For example, the components could be worn by a person or placed on a person and used to assist a patient in a flexion and extension task, while the system monitors and analyzes the movement, and helps a patient complete a recovery protocol. These active components may or may not be controlled by the system, or be independent and/or have their control signals integrated with the system. Furthermore, the systems could be controlled by active or passive feedback between the different components. Furthermore, these devices can also provide data that can be used by the CPU to assess patient movement characteristics such as for example movement measurement data, trigger information, synchronization information, and/or timing information. These active components can also be used to provide stimuli to the patient during task assessment.

### Diagnosis

The system and the alternative embodiments described herein can be used diagnostically, such as to aid in or to provide the diagnosis of a disease or disorder, or to aid in or provide the differential diagnosis between different diseases or disorder states. The system can also be used as a diagnostic tool, where a diagnosis is made based on the response to a therapy as demonstrated from the motion analysis system, for example giving a suspected Parkinsonian patient a treatment of dopamine and assessing the patient's response to the drug with the motion analysis system. The system could also be used to stratify between different disease states, such as for example using the motion analysis system to determine what type of progressive supra nuclear palsy (PSP) a PSP patient has and/or to determine the severity of a disease or disorder. The system can be used to provide a diagnosis with or without the input of a clinician, and in certain embodiments the system can be used as a tool for the clinician to make a diagnosis.

In certain embodiments of the diagnostic system, the system uses a reference set of data to which a subject is compared in order to make a diagnosis or assessment of the subject. The reference set, stored on the CPU or remotely on a server operably coupled to the CPU, includes data of normal healthy individuals and/or individuals with various ailments of various ages, genders, body type (e.g., height, weight, percent body fat, etc.). Those healthy individuals and/or individuals with various ailments have been analyzed using the motion analysis system of the invention and their data is recorded as baseline data for the reference data set (in alternative embodiments, a reference set can be developed by modeling simulation motion data and/or a reference set could be developed from a mathematical model developed based on the analysis of assessments of healthy individuals and/or patients). The reference set of data could be based on previous measurements taken from the patient currently being assessed. A test subject is then evaluated using the motion analysis system of the invention and their kinematic and/or kinetic information is compared against the appropriate population in the reference set, i.e., the test subject data is matched to the data of a population within the reference set having the same or similar age, gender, and body type as that of the subject. The difference, if any, between the test subject's kinematic and/or kinetic information as compared to that of the reference data set allows for the assessment and/or diagnosis of a movement disorder in the subject. Typically, at least a 25% difference (e.g., 30%, 35%, 40%, 45%, 50%, 60%, 70%, 80%, 90%, 95%, 95%, or 99%) between the kinematic and/or kinetic information of the subject and that of the reference data set is an indication that the subject has a movement disorder. The greater the difference between the kinematic and/or kinetic information of the subject and that of the reference data set allows for the assessment of the severity or degree of progression of the movement disorder. For example, a subject with at least 50% difference (e.g., 55%, 60%, 70%, 80%, 90%, 95%, 95%, or 99%) between their kinematic and/or kinetic information and that of the reference set would be considered to have a severe form of the movement disorder. In certain diagnostic tests, just the presence of a characteristic, for example a Babinski sign, would be enough to demonstrate a patient has an ailment, which for a Babinski would demonstrate an upper motor neuron deficit in the system. In certain diagnostic evaluations, smaller differences can be demonstrated, such as for example to track a patients progress to a therapy (such as when comparing a patient's motion analysis results previous motion analysis results from a previous exam of the patient). Furthermore multiple small differences (e.g., less than 25%), for example in at least two criteria, between a tested patient and a data set can be used to make a probabilistic diagnosis that a patient suffers from a disorder (for example, in certain alternative embodiments, multiple changes, with changes as small as 1%, could be used to make a statistical model, that can have a predictive capabilities with high probability that a disease is present (such as for example with 80%, 90%, 95%, 99%, 99.9% and 100% probability)—for example: a statistical model based on 10 different movement task characteristics could be assessed which makes a diagnosis based on a weighted probabilistic model, a disease diagnosis model based on derived results or grouped results (e.g., positive presence of a Babinski sign when 99 other tested criteria were not met, would still be an indication of an upper motor neuron disease), and/or model based on patient history and a result(s) derived from the motion analysis system while patients are performing a movement or set of movement tasks).

In certain embodiments, the CPU can contain and/or be connected to an external database that contains a set of disease characteristics and/or a decision tree flow chart to aid in or complete the diagnosis of a disease. In certain embodiments the system can take information in about the patient demographics and/or history. In certain embodiments, the CPU might direct a clinician to perform certain tests based on a patients history and chief complaint or the clinician could have the choice to completely control the system based on their decisions. The test plan (i.e., planned tasks to be conducted and subsequent analysis) can be modified throughout the entire patient exam, based on results gathered from an ongoing exam (such as for example based on a probabilistic decision derived from the motion analysis system measured patient movement characteristics and CPU analysis) and/or clinician interaction. The system could be programed to conduct a part of an exam, such as a cranial nerve exam, or a focused exam relative to an initial presentation of symptoms or complaint of a patient, such as for example a motor exam tailored by the system to compare PSP and Parkinson's Disease, and/or other potential movement disorders.

For example, a patient might come to a clinic with complaints of slowed movement and issues with their balance including a history of falls. The patient could be fitted with a number of motion analysis sensors, such as accelerometer and gyroscopes, and be asked to perform a number of tasks while in view of an image capture system, and the data from these measurements are processed by a CPU to aid in or provide a patient diagnosis. In this example herein the CPU might process demographic information about the patients (e.g., 72 years, male) and that the patient has a history of falls, and is presenting with a chief complaint of slowed movement and complaints of balance problems. Based on this information the system could recommend a set of tasks for the patient to complete while being analyzed by the system, and/or a clinician can direct the exam (for example based on a epidemiological data set of potential patient diagnoses from a reference set).

In this example, the doctor first instructs the patient to perform a number of tasks, such as a flexion and extension task or a combined movement task, to determine movement characteristics such as the speed, smoothness, and/or range of movement that they are moving at during the task, which is compared to a reference set of data (e.g., matched healthy individuals and/or patients suffering from various ailments). By using the motion analysis system, with the data from the body motion sensors and the image capture system, the CPU could complete the analysis exemplified as above, and compare this data to matched (e.g., age, sex, etc.) subjects who performed the same tasks. The CPU directed comparison to reference data could be made just compared to healthy individuals, to patients suffering from a pathology or pathologies, and/or both. The system analysis of the patient task performance could establish that the example patient has slowed movements (i.e., bradykinesia), indicating the potential for a hypokinetic disorder, and demonstrate that the symptoms are only present on one side of the body (note this example of patient symptoms provided herein is just exemplary and not meant to be limiting but provided to demonstrate how this diagnostic embodiment of the device could be used with an example patient).

Next, while being assessed by the motion analysis system, the patient could be asked to perform a number of additional movement tasks to assess for tremor and/or additional quality measures of movement (such as by using the system as exemplified above). This could for example establish that the patient has no evidence of postural, resting, and/or action tremor (aka kinetic tremor) relative to matched healthy subjects or patients suffering from tremor pathologies (e.g., the example patient demonstrates insignificant signs of increased power in frequency bands indicative of abnormal tremors as determined by the CPU by comparing the motion analysis system results with a reference data set).

In certain alternative embodiments the system could be designed to assess and compare tremors of different diseases such as for example Parkinsonism, multiple sclerosis, cerebellar tremor, essential tremor, orthostatic tremor, dystonic tremor, and/or enhanced physiological tremors (with each other and/or with a normal physiological tremor). As above, the tremor could be correlated with numerous conditions, such as body position, joint position and/or movement, for the diagnosis of a movement disorder.

Next while being assessed by the motion analysis system, the patient could be asked to stand still and have the posture analyzed by the system, such as by using the system as exemplified above. In this case the system analysis of the patient could for example demonstrate that the patient has a very subtle posture abnormality where they are leaning backwards while standing up relative to matched healthy subjects (indicative of rigidity of the upper back and neck muscles seen in certain pathologies in matched patients, such as those with PSP).

Next while being assessed by the motion analysis system, the patient could stand on a force plate and have their balance analyzed in number of different states (e.g., eyes open, eyes closed, feet together, feet apart, on one foot, and/or with a clinician provided perturbation (e.g., bump)), such as by using the system as exemplified above. In this case the system analysis of the patient could for example demonstrate a lack of stability (e.g., large disturbances in their center of gravity) and demonstrate a positive Romberg sign relative to healthy matched subjects and being indicative of matched patients suffering from various pathologies that negatively effect their balance (such as Parkinsonism).

Next while being assessed by the motion analysis system, the patient could then be asked to walk along a 10 meter path, turn around, walk another 10 meters back to the starting point. As described above, the patient's gait and/or posture characteristics could be analyzed and/or compared relative to matched subjects. For example in this patient, it could be shown with the motion analysis system that the patient has a slower average gait speed and a smaller stride length than a typical matched healthy subject (furthermore it might be shown that their stride and gait characteristics were more effected on one side of the body than the other, which was comparable with their bradykinesia symptoms, and potentially indicative of Parkinsonism given the other data analyzed).

Next while being assessed by the motion analysis system, the clinician could also manually manipulate the patient's joint(s), by providing a fixed, measured, random, and/or calculated force to move a joint of the patient. This manipulation could be done while asking the patient to be passive, to resist, and/or to move in a certain manner. This manipulation could be accomplished by an external or additional integrated system, such as by a robot. The motion analysis suite could assess the joint displacement characteristics to the clinician provided manipulation. This information could be used as a measure of the patient's rigidity. There are a number of ways the motion analysis system and alternative embodiments could assess rigidity. For example, the motion analysis suite can determine the response of the joint to the clinician provided manipulation by assessing patterns of movement such as explained above (for example the magnitude of movement along a path length, directional response, power in response), or whether the trajectory or the joint displacement is continuous and smooth such as for example whether it might show a cogwheel (which presents as a jerky resistance to passive movement as muscles tense and relax) or lead-pipe (the body won't move; it's stiff, like a lead pipe) rigidity pattern. In certain embodiments, the system can be used to determine the force or characteristics of the movement perturbing the joint and the response of the joint to the manipulation, such as by using the accelerometer data of magnitude and relative acceleration direction (where in certain embodiments the exact direction in the patients' coordinate system are determined by the camera) and/or a calculation of mass of the joint (for example, the image capture device could be used to provide dimension information about the joint being moved (e.g., arm and wrist information in an elbow example), and with that information a calculation of the mass of the moved joint could be determined based on typical density information of the limb). In certain embodiments, the acceleration of the perturbation movement (i.e., the manipulation movement of the joint) could be used in lieu of force (for example, one could determine the response of a joint to an external acceleration). In certain embodiments, the force or movement characteristics that the clinician provides to manipulate or perturb the patient's joints can also be determined by having the clinician wear at least one external motion sensor (such as an accelerometer) and/or be analyzed by the motion analysis system where in certain embodiments they are also assessed by the motion capture device. Additionally, the force to manipulate a joint provided can be measured by a separate system and/or sensor and provided real-time or/at a later point to the system for analysis. In this example patient, the patient could show an absence of rigidity in the arms and legs (e.g., throughout the upper and lower limbs) as assessed by the motion analysis system.

Note, as described in the assessment and analysis of a patient's potential rigidity, in certain embodiments, the clinician could wear at least one external motion sensor and/or place at least one on or in an external instrument used as part of the exam for any part of the patient analysis. Such, as for example one could assess a patient's reflexes to an accelerating hammer, with fixed mass and shape characteristics, which has an accelerometer in it. In this example, the clinician could note normal joint reflexes in the upper and lower limb as assessed by the motion analysis system.

Next while being assessed by the motion analysis system, the patient might also be asked to hold both arms fully extended at shoulder level in front of him, with the palms upwards, and hold the position, either in a normal state, with their eyes closed, and/or while the clinician and/or system provides a tapping (such as through a active system component in certain system embodiments) to the patient's hands or arms. If the patient is unable to maintain the initial position the result is positive for pronator drift, indicative of an upper motor neuron disease and depending on the direction and quality of the movement the system could determine the cause (such as from a cerebellar cause, such as for example when forearm pronates then the person is said to have pronator drift on that side reflecting a contra-lateral pyramidal tract lesion. A lesion in the cerebellum usually produces a drift upwards, along with slow pronation of the wrist and elbow). The system could complete the analysis of the movements and comparison to a reference data set as above, and demonstrate that the example patient shows no differences in pronator drift relative to matched healthy subjects.

Next while being assessed by the motion analysis system, the patient might then be asked to remove their shoe and the clinician might place an accelerometer on the patient's big toe (if it was not used for any of the previous tasks). The physician could than manually run an object with a hard blunt edge along the lateral side of the sole of the foot so as not to cause pain, discomfort, or injury to the skin; the instrument is run from the heel along a curve to the toes (note the motion analysis system could also automate this with an active component). The accelerometer (and/or other motion sensor) and image capture device can determine whether a Babinski reflex is elicited in this patient (The plantar reflex is a reflex elicited when the sole of the foot is stimulated with a blunt instrument. The reflex can take one of two forms. In normal adults the plantar reflex causes a downward response of the hallux (flexion), which could be recorded with the system. An upward response (extension) of the hallux is known as Koch sign, Babinski response or Babinski sign, named after the neurologist Joseph Babinski. The presence of the Babinski sign can identify disease of the spinal cord and brain in adults, and also exists as a primitive reflex in infants)). The system could complete the analysis of the movements and comparison to a reference data set as above, and demonstrate that the example patient did not have a definitive Babinski.

Next while being assessed by the motion analysis system, the patient might then be given a cognitive exam (such as for example a mini mental state exam, General Practitioner Assessment of Cognition (GPCOG), Mini-Cog, Memory Impairment Screener, Language Assessment Screener, Wisconsin Card Sorting Test, Dementia Rating Scale, Hooper Visual Organization Test, Judgment of Line Orientation-Form V, Scale of Outcomes of Parkinson Disease-Cognitive, the Neuropsychiatry Inventory, and/or comparable instruments), to assess the patients' cognitive level or assess if there are any other deficits, which in certain embodiments could be conducted via components connected to and controlled by the motion analysis system CPU. The system could also gather data from the patient such as history and/or other symptom information not gathered at the onset of the exam but determined important as a result of the CPU analysis based on data gathered as part of the patient exam (for instance whether this patient had sleep disturbances or a history of hallucinations), which could be determined from simple questions, or by connecting the motion analysis system to other systems which can assess a patients sleep characteristics (e.g., REM sleep disturbances). For this example, as could be determined by the motion analysis system (connected to additional system components that provided a cognitive exam), this example patient could demonstrate no cognitive abnormalities that indicate severe dementia or cognitive decline compared to the CPU analyzed reference sets.

At this stage for this example, the clinician has analyzed the patient with the motion analysis system and the patient demonstrates positive signs for asymmetric bradykinesia, gait abnormalities (with issues more pronounced on one side), a slight posture abnormality indicative of rigidity in the neck and upper back but no pronounced rigidity in the peripheral joints, and poor general balance with a positive Romberg sign. Based on the results of the motion analysis system, the system and the doctor indicate that the patient has early stage Parkinson's Disease or early PSP. The doctor sends the patient home with a prescription for 1-dopa and tells the patient to come back in 8 to 12 weeks (or a typical period for a patient who is responsive to the drug to begin responding to the medication).

When the patient returns, the clinician confirms that the patient has taken their course of medicine and repeats the exam with the motion analysis system as above. Unfortunately, the system reveals that patient has the same symptoms as above, did not respond to the 1-dopa, but now presents with a more pronounced symptoms. The motion analysis system makes a definitive diagnosis of early stage PSP and the doctor begins treating the patient with brain stimulation and tracking the patient with the motion analysis system.

In alternative embodiments, the PSP patient (or any patient being examined) could have had their eyes examined at any stage during the exam. For example on the follow-up appointment, an eye tracking system could have been used to analyze the patients vertical and horizontal gaze and specifically been used to assess whether there was a recording of restricted range of eye movement in the vertical plane, impaired saccadic or pursuit movements, abnormal saccadic or smooth pursuit eye movements, and/or other visual symptoms (the recording of other visual symptoms not explained by the presence of gaze palsy or impaired saccadic or pursuit movements, which could evolve during a PSP disease course. Symptoms include painful eyes, dry eyes, visual blurring, diplopia, blepharospasm and apraxia of eyelid opening). This eye tracking could be conducted by a connected to and/or integrated component of the motion analysis system, and the CPU analysis of this eye data, by itself and/or in combination with the other motion data could be compared to a reference set of healthy and patient performances to make the diagnosis of PSP or some other ailment.

In further alternative embodiments, the system could be connected with sensors that evaluate a patients autonomic function such as for example urinary urgency, frequency or nocturia without hesitancy, chronic constipation, postural hypotension, sweating abnormalities and/or erectile dysfunction (which in certain embodiments could also be determined through an automated system of questions answered by the patient).

In further embodiments, the motion analysis system and connected components could be used to analyze a patients speech patterns and voice quality (such as for example through facial recognition, sound analysis, and/or vocal cord function as measured with accelerometers).

In alternative embodiments, the CPU can be programmed to analyze and track the drug history and status of the patient and be used in making diagnostic decisions or to develop more effective drug (or other therapy) dosing regimens.

In another example, another patient comes in to a clinicians office with complaints of general slowness of movement. The patient could be fitted with a number of motion analysis sensors, such as accelerometer and gyroscopes, and be asked to perform a number of tasks while in view of an image capture system, while data from these measurements are processed by a CPU to aid in or provide a patient diagnosis. The patient completes the same test as above, and demonstrates cogwheel rigidity, slowed velocity of movement, pronounced action tremor, pronounced resting tremor, pronounced postural tremor, all of which are more pronounced on the right side of the body in comparison to a healthy reference set. The system makes a diagnosis of classical Parkinson's disease.

In certain embodiments, the system would have a defined neural exam outline to conduct, based on a cranial nerve exam, a sensory exam, a motor strength exam, a sensory exam, a coordination exam, autonomic function analysis, reflexes, and/or cognitive exams (such as for example exams such as discussed in “Bates' Guide to Physical Examination and History-Taking” by Lynn Bickley MD (November 2012)).

For example, the motion analysis system could be designed to assess a patient's cranial nerves. In the first set of tasks the system is used to assess the visual acuity and eye motion of the patient. A visual monitor could be connected to the CPU, which controls visual stimuli sent to the patient, and the image capture device and/or eye tracking system could be used to record the patient movements and eye characteristics to determine the function of cranial nerves 2, 3, 4, and 6. In certain embodiments a sound recording and production device could also provide and record eye exam directions and responses (e.g., record the response from reading a line of letters, provide instructions to look upwards or to follow a light on a screen). The image capture component of the system, and potentially facial recognition software, and/or face and shoulder mounted motion sensor could be used to assess a patients ability preform facial and shoulder movements which could help in assessing the function of cranial nerve 5, 7, 9, and 11 where the patient could be instructed to complete various movements, such as example movements demonstrated to a patient on a monitor. For example such an assessment could be used to help determine and diagnose if a patient had a stroke, where with a stoke (upper motor neuron injury) a patient might have a droopy mouth on one side and a spared forehead with the ability to raise their eyebrows (compared to another disorder such as Lyme disease where the forehead is not spared and a patient can't raise their eyebrow). In certain embodiments, the system could implement active stimuli generating components, such as for example where components could generate light touch stimuli on the location such as the forehead or cheek to assess the sensory component of the 5^{th} and 7^{th} cranial nerves, where the system could provide the stimuli to the patient and assess whether they sense the stimuli, relative to a certain location on their face as determined by the CPU and data from the image capture component (such as for example via visual feedback from the patient). The system could provide sound stimuli to assess the 8^{th} cranial nerve, based on feedback responses from the patient as to how well they hear certain stimuli. To assess the movements generated the 9^{th} and 10^{th} cranial nerve, using the system such as described above, the patient could be instructed to swallow and say “ah” and additionally assess whether their voice was horse (such as through additional sound recording and analysis methods outlined above). And finally for an evaluation of the 12the cranial nerve the system could assess the patient as they move their tongue in various directions and through various movements (following the methods and analysis described above).

As another example the motion analysis system could analyze the coordination of a patient, such as for example conducting tests such as those outlined above or other tests such as assessing things such as rapid alternating movements, flipping the heads back and forth, running and/or tapping the finger to the crease of the thumb. These tasks would be completed and analyzed as described above.

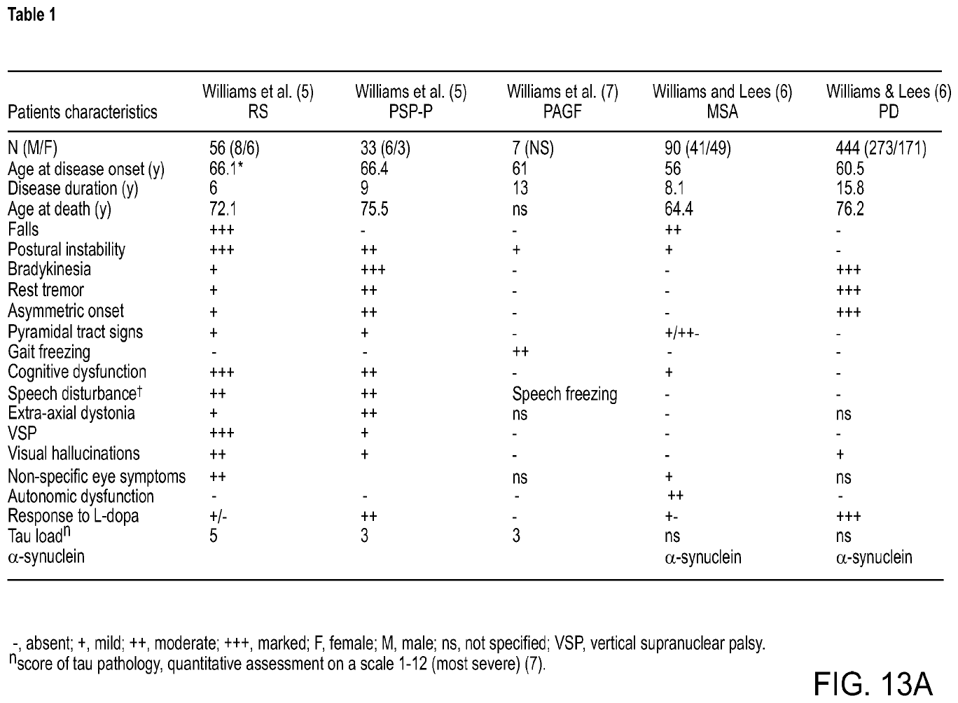

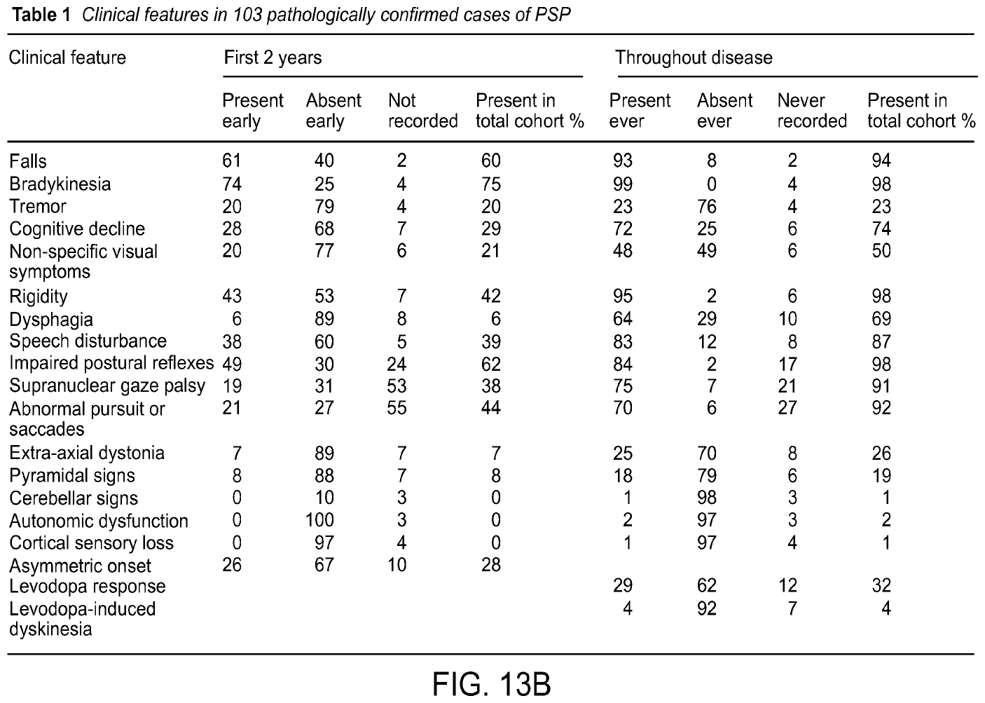

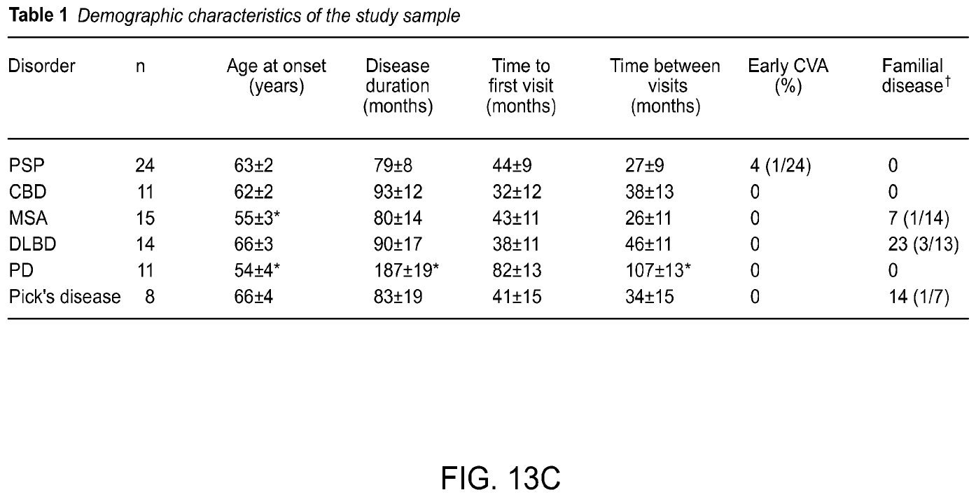

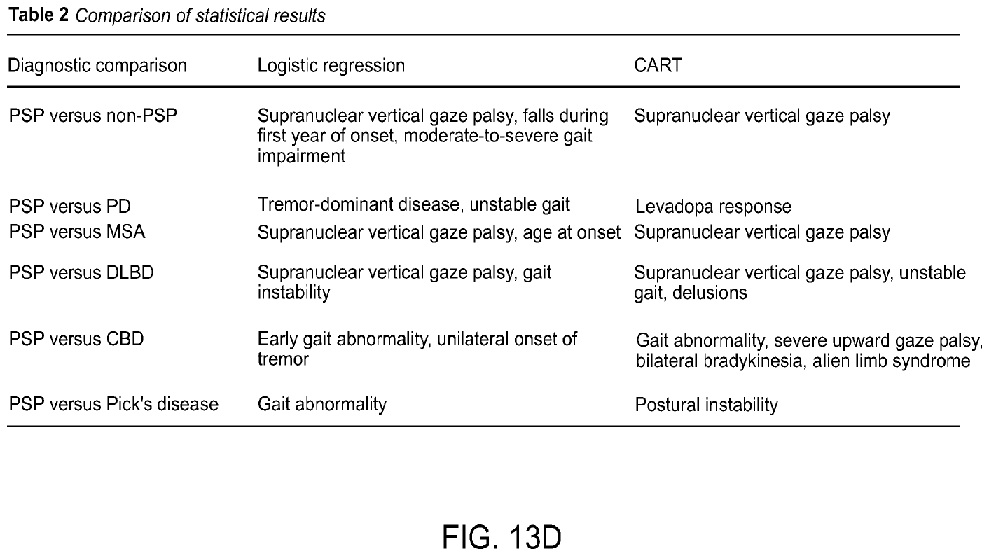

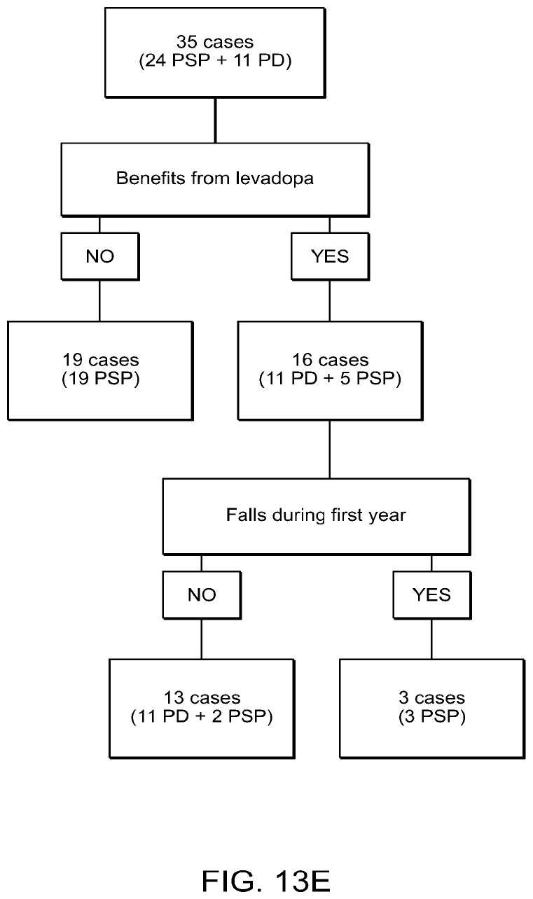

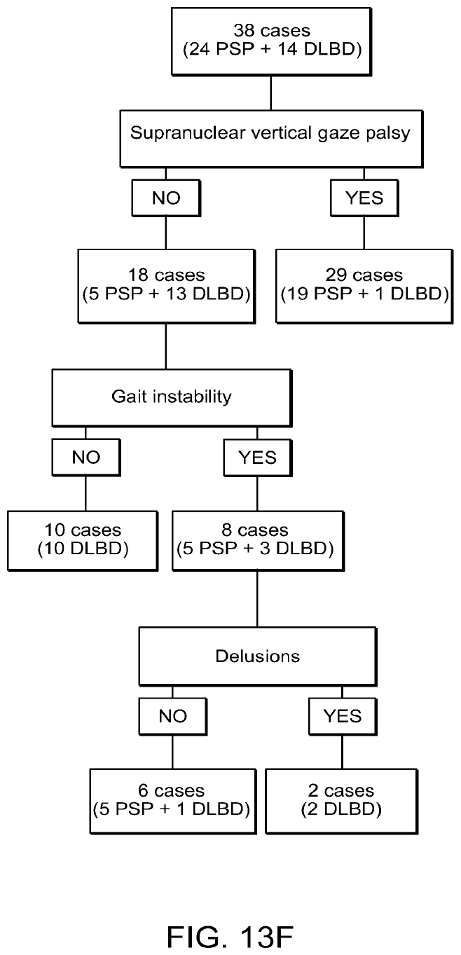

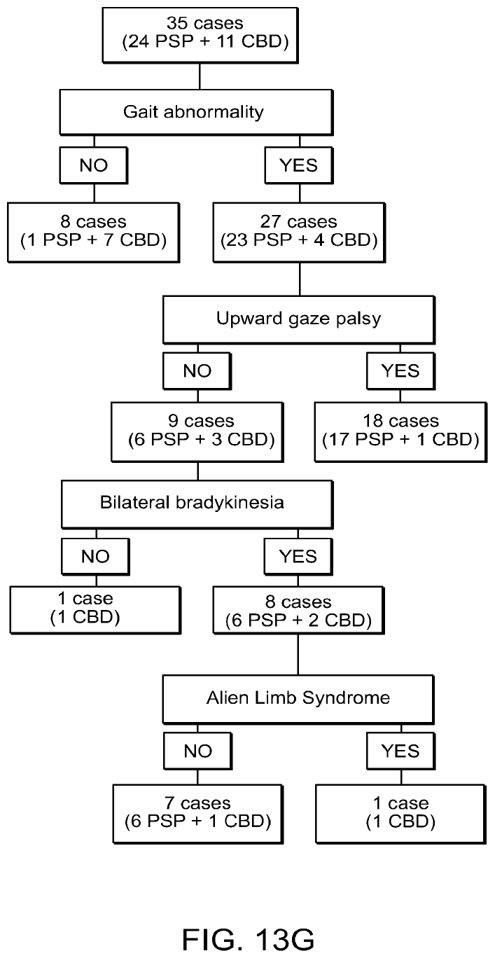

The system could have a focused neural exam based on disease characteristics that serve as part of a differential diagnosis, such as for example the could conduct a specific sub-set of a complete neural exam based on preliminary information provided by the patient. For example, a patient who's chief complaints are slowness of movement, balance abnormalities, and a history of falls could be provided a focused exam like above in the example patient diagnosed with PSP. The exam flow could be based on patient characteristics determined from across a number of previous cases, as could similarly the diagnostic criteria that the CPU uses to determine the disease state of the patient. For example, in the above PSP diagnosis example the diagnosis could be made based on defined criteria such as in FIG. 13A which is from “Liscic R M, Srulijes K, Gr€oger A, Maetzler W, Berg D. Differentiation of Progressive Supranuclear Palsy: clinical, imaging and laboratory tools. Acta Neurol Scand: 2013: 127: 362-370.” and/or FIG. 13B which is from “Williams et al. Characteristics of two distinct clinical phenotypes in pathologically proven progressive supranuclear palsy: Richardson's syndrome and PSP-parkinsonism. Brain (2005), 128, 1247-1258.” Table 1 in FIG. 13 A shows demographic and early clinical features of PSP disorders (Richardson syndrome (RS), PSP-parkinsonism (PSP-P), pure akinesia with gait freezing (PAGF) compared with Parkinson's disease (PD) and multiple system atrophy (MSA) in cohorts of pathologically confirmed cases. The age at disease onset and disease duration in Table 1 in FIG. 13A are means. Early dysarthria or dysphagia are included in speech disturbances in Table 1 in FIG. 13A.

The motion analysis system could implement: a diagnostic flow chart based on previous studies to determine a diagnosis; a weighted decision tree based on a neuro-exam based flow chart; follow the exam and diagnostic flow of statistical studies of a disease such as could be exemplified in FIG. 13C-13G from “Litvan et al. Which clinical features differentiate progressive supranuclear palsy (Steele-Richardson-Olszewski syndrome) from related disorders? A clinicopathological study. Brain (1997), 120, 65-74.” a statistical prediction based on the patient criteria measured by the motion analysis system and a look up table of patient characteristics demonstrated in previous populations of patients; a probabilistic model based on past patient disease characteristics (e.g., probability of having disease given symptoms, etc) across multiple disease states; and/or use prediction models such as those described in “The Statistical Evaluation of Medical Tests for Classification and Prediction (Oxford Statistical Science Series) by Margaret Sullivan Pepe (December 2004)”, “Clinical Prediction Models: A Practical Approach to Development, Validation, and Updating (Statistics for Biology and Health) by by Ewout Steyerberg (October 2008)”, “Statistical Methods in Diagnostic Medicine by Xiao-Hua Zhou, Nancy A. Obuchowski, Donna K. McClish (March 2011)” the content of each of which is incorporated by reference herein in its entirety.

In Table 1 provided in FIG. 13C, values are given as mean ±SEC, PSP=progressive Supranuclear palsy; CBD=corticobasal degeneration, MSA=multiple system atrophy, DLBD=diffuse Lewy body disease, PD=Parkinson's disease, and Early CVA=stroke before the first visit. The starred (*) values have P<0.05 for the difference from other disorders studied. Familial disease was defined as at least on relative with a similar disorder. In Table 2 provided in FIG. 13D, PSP=progressive Supranuclear palsy, PD=Parkinson's disease, MSA=multiple system atrophy, DLBD=diffuse Lewy body disease, CBD=corticobasal degeneration, and CART =classification and regression tree analysis.

### Stimulation

As already mentioned above, systems and methods of the invention can be used with stimulation protocols. Any type of stimulation known in the art may be used with methods of the invention, and the stimulation may be provided in any clinically acceptable manner. For example, the stimulation may be provided invasively or noninvasively. Preferably, the stimulation is provided in a noninvasive manner. For example, electrodes may be configured to be applied to the specified tissue, tissues, or adjacent tissues. As one alternative, the electric source may be implanted inside the specified tissue, tissues, or adjacent tissues. Exemplary apparatuses for stimulating tissue are described for example in Wagner et al., (U.S. patent application numbers 2008/0046053 and 2010/0070006), the content of each of which is incorporated by reference herein in its entirety.

Exemplary types of stimulation include mechanical, optical, electromagnetic, thermal, or a combination thereof. In particular embodiments, the stimulation is a mechanical field (i.e., acoustic field), such as that produced by an ultrasound device. In other embodiments, the stimulation is an electrical field. In other embodiments, the stimulation is an magnetic field. Other exemplary types of stimulation include Transcranial Direct Current Stimulation (TDCS), Transcranial Ultrasound (TUS), Transcranial Doppler Ultrasound (TDUS), Transcranial Electrical Stimulation (TES), Transcranial Alternating Current Stimulation (TACS), Cranial Electrical Stimulation (CES), Functional Electrical Stimulation (FES), Transcutaneous Electrical Neural Stimulation (TENS), or Transcranial Magnetic Stimulation (TMS). Other exemplary types include implant methods such as deep brain stimulation (DBS), microstimulation, spinal cord stimulation (SCS), and vagal nerve stimulation (VNS). In certain embodiments, stimulation may be provided to muscles and/or other tissues besides neural tissue. In other embodiments, the stimulation source may work in part through the alteration of the nervous tissue electromagnetic properties, where stimulation occurs from an electric source capable of generating an electric field across a region of tissue and a means for altering the permittivity and/or conductivity of tissue relative to the electric field, whereby the alteration of the tissue permittivity relative to the electric field generates a displacement current in the tissue. The means for altering the permittivity may include a chemical source, optical source, mechanical source, thermal source, or electromagnetic source.

In other embodiments, the stimulation is provided by a combination of an electric field and a mechanical field. The electric field may be pulsed, time varying, pulsed a plurality of time with each pulse being for a different length of time, or time invariant. Generally, the electric source is current that has a frequency from about DC to approximately 100,000 Hz. The mechanical field may be pulsed, time varying, or pulsed a plurality of time with each pulse being for a different length of time. In certain embodiments, the electric field is a DC electric field.

In other embodiments, the stimulation is a combination of Transcranial Ultrasound (TUS) and Transcranial Direct Current Stimulation (TDCS). Such a combination allows for focality (ability to place stimulation at fixed locations); depth (ability to selectively reach deep regions of the brain); persistence (ability to maintain stimulation effect after treatment ends); and potentiation (ability to stimulate with lower levels of energy than required by TDCS alone to achieve a clinical effect).

In certain embodiments, methods of the invention focus stimulation on particular structures in the brain that are associated with arthritic pain, such as the somatosensory cortex, the cingulated cortex, the thalamus, and the amygdala. Other structures that may be the focus of stimulation include the basal ganglia, the nucleus accumbens, the gastric nuclei, the brainstem, the inferior colliculus, the superior colliculus, the periaqueductal gray, the primary motor cortex, the supplementary motor cortex, the occipital lobe, Brodmann areas 1-48, the primary sensory cortex, the primary visual cortex, the primary auditory cortex, the hippocampus, the cochlea, the cranial nerves, the cerebellum, the frontal lobe, the occipital lobe, the temporal lobe, the parietal lobe, the sub-cortical structures, and the spinal cord. Stimulation and the effects of stimulation on a subject can be tuned using the data obtained from this system. Tuning stimulation and its effects are discussed, for example in U.S. patent application Ser. No. 14/335,282, the content of which is incorporated by reference herein in its entirety. Furthermore, the motion analysis system could be used as part of a DBS stimulation parameter tuning process.

In certain embodiments, stimulation and the motion analysis system can be coupled to aid in the diagnosis of a disorder. For example, brain stimulation can be applied to a specific brain area that is expected to be affected by a disease being tested for. The response of joints that are connected to the brain area can be assessed by the motion analysis system. And the motion analysis system analysis of these movements in conjunction with the stimulation response could be used to aid in the diagnosis of a disease (for example, if a patient as being tested for a lesion to the right primary motor cortex hand area of the patient under study, stimulation to the left primary cortex is expected to generate a diminished response of hand motion in the presence of a lesion).

A combined stimulation and motion analysis system could also be used to determine mechanisms of a disease or disorder, and/or methods for more appropriately treating the disease or disorder. For example, we found that stimulation to a Parkinson's Disease patient's primary motor cortex had a benefit on certain symptoms of the disease as demonstrated by the motion analysis system, and in turn we could look at those responses to stimulation to compare their differential response to determine additional therapies and explore fundamental mechanisms of the disease (such as for example comparing the differential effect of stimulation on a patient's balance with their eyes open and closed, and using this and other data to determine the impact of the disease on the patient's direct and indirect pathway, and then in turn adapting the location of stimulation based on the motion analysis data results and knowledge of these pathways to target a more effective area of the brain).

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein.

### EXAMPLES

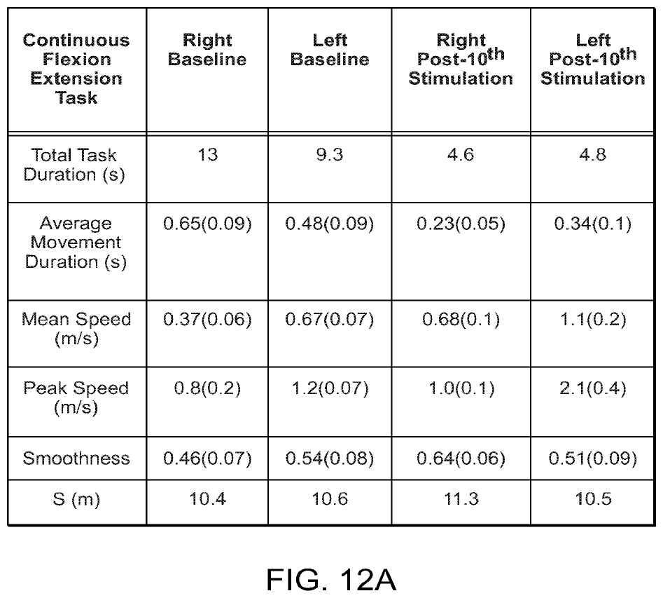

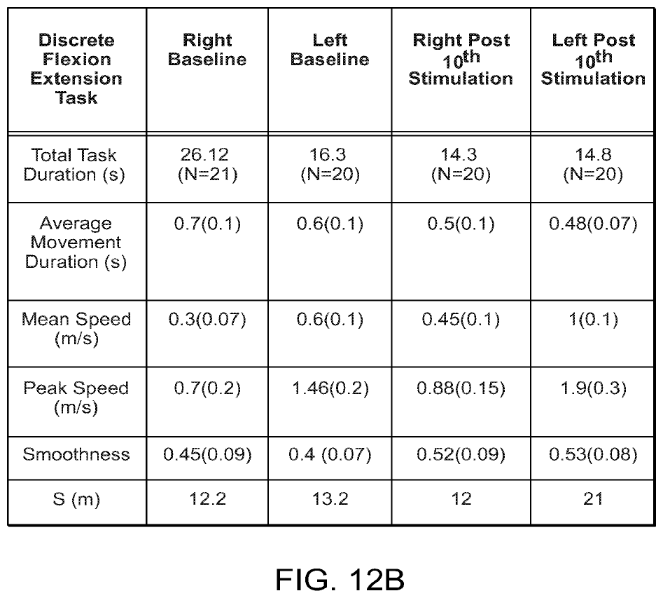

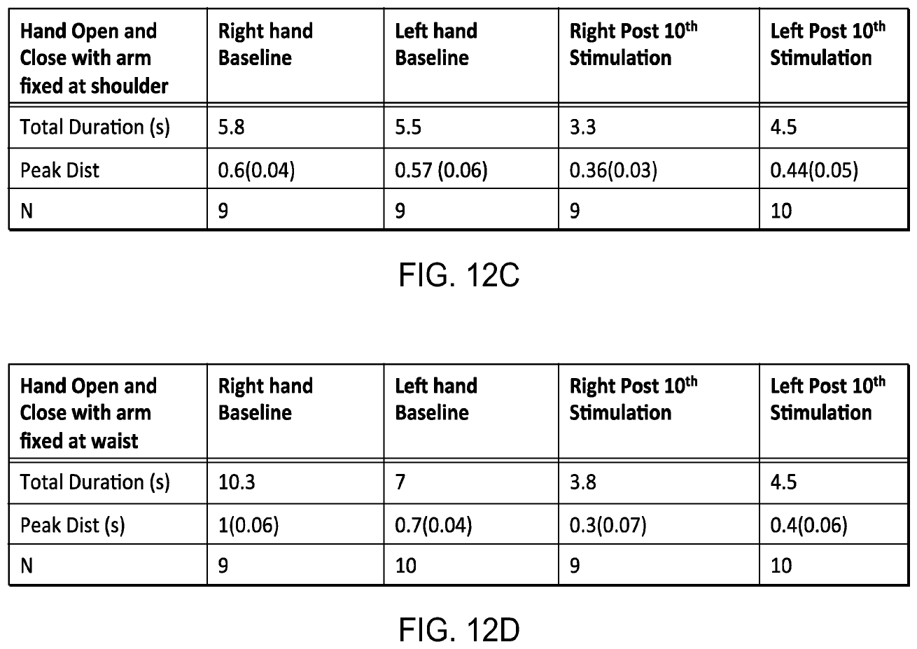

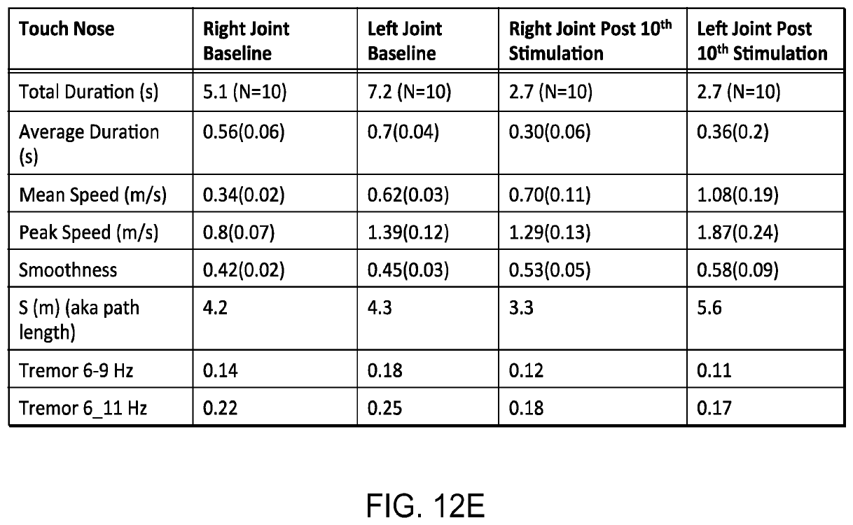

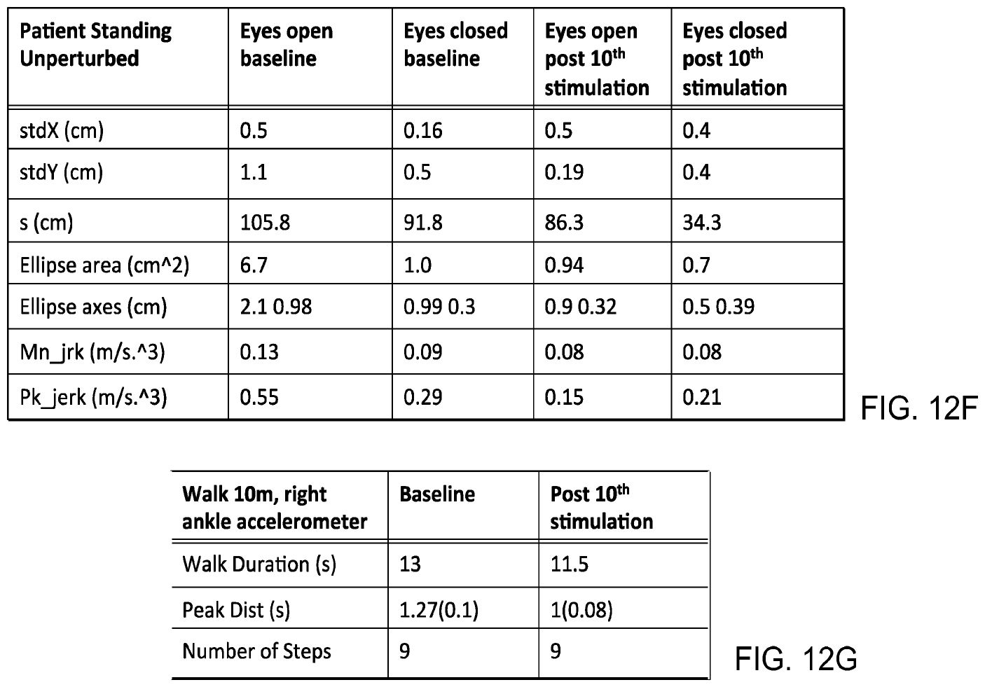

We implemented a stimulation system that provided electrosonic stimulation (ESStim™) to a Parkinson's disease patient's primary motor cortex 5 days a week for twenty minutes a day over 2 weeks (10 days total). We had the patient perform a bradykinesia test where the patient was asked to perform 10 arm flexion-extension movements as fast as possible while the patient was analyzed with the motion analysis system (the test and analysis are described above; where, baseline (i.e., before any stimulation was provided) measurements from this patient are provided in FIG. 6 and used to specifically describe the implementation of the analysis used for this task as described above). We conducted these tests at baseline (i.e., before any stimulation was provided), throughout the course of stimulation, and for weeks following stimulation. The following metrics were determined: movement mean speed (mean value of speed), movement peak speed (peak value of speed), movement duration: difference between offset of movement and onset of movement, movement smoothness (mean speed/peak speed). Also calculated is the path length of the trajectory of the wrist joint (distance traveled in 3D space). Results for this task, from the 10^{th} day stimulation and the baseline of the patient are provided in FIG. 12A.

We had the patient perform a bradykinesia test where the patient was asked to perform 10 arm flexion-extension movements. After each flexion or extension movement, the subject is asked to stop. The movements were performed as fast as possible (the test and analysis are described above, and FIG. 7 was based on the baseline information for this patient). Results for this task, from the 10^{th} day stimulation and the baseline of the patient are provided in FIG. 12B.

We had the patient perform a task where they were asked to perform 10 hand opening and closing movements, as fast as possible, while the hand was positioned at the shoulder (the test and analysis are described above, and FIG. 8 was based on the baseline information for this patient). Results for this task, from the 10^{th} day stimulation and the baseline of the patient are provided in FIG. 12C. In FIG. 12D, we provide a similar set of results for the patient where the task was performed with the hand positioned at the waist.

We had the patient perform a task where they were asked to combine the movements (flexion followed by hand opening/closing followed by extension followed by hand opening/closing) 10 times as fast as possible. Analysis based on the combined analysis of the individual motions was completed, and similar improvements were shown. For example, the patient took 26.6 and 23.6 seconds to perform the tasks at baseline (for the right and left joints respectively) and 23.6 and 20.6 seconds for the tasks after the 10^{th} stimulation session.

We had the patient perform a task where they were asked to touch their nose with their index finger, as completely as possible, 5 times (the test and analysis are described above, and FIG. 9 was based on the baseline information for this patient). The following metrics are the final output for this test: total duration of test; number of movements actually performed; movement mean speed (mean and standard deviation across all movements); movement peak speed (mean and standard deviation across all movements); movement duration (mean and standard deviation across all movements); movement smoothness (mean and standard deviation across all movements); path length; tremor in the range 6-9 Hz; tremor in the range 6-11 Hz. Results for this task, from the 10^{th} day stimulation and the baseline of the patient are provided in FIG. 12E.

We had the patient perform a task where the patient's posture and/or balance characteristics are assessed. The patient was asked to stand on a force plate while multiple conditions are assessed: eyes open and eyes closed. During measurements with eyes open or closed, the subject was simply asked to stand on a force plate. The test and analysis are described above, and FIG. 10 was based on the baseline information for this patient. Results for this task, from the 10^{th} day stimulation and the baseline of the patient are provided in FIG. 12F.

We had the patient perform a task for assessing gait and/or posture, where the patient was asked to walk 10 meters. The test and analysis are described above, and FIGS. 11A and 11B was based on baseline information for this patient. Results for this task, for the right ankle accelerometer and associated measures, from the 10^{th} day stimulation and the baseline of the patient are provided in FIG. 12G.

In other implementations, such as for example where stimulation is given to other patients who are less responsive to stimulation, for patients given less stimulation (i.e., a lower dose of stimulation), or for less effective types of stimulation the motion analysis system we describe herein can still be effective in demonstrating the effects of stimulation. Similarly in other implementations, such as for example where stimulation is given to other patients who are more responsive to stimulation, for patients given more stimulation (i.e., a larger dose of stimulation), or for more effective types of stimulation the motion analysis system we describe herein can still be effective in demonstrating the effects of stimulation.

For example, in another Parkinson's Disease patient, receiving the same stimulation protocol, and assessed with the bradykinesia test where the patient was asked to perform 10 arm flexion-extension movements as fast as possible while the patient was analyzed with the motion analysis system. For their right arm they demonstrated a baseline total time of task of 11.58 seconds, and average movement duration of 0.568 seconds, a mean speed of movement of 0.558 m/s, and a peak speed of 1.201 m/s. Following the 10^{th} simulation they demonstrated a total time of task of 13.3 seconds, and average movement duration of 0.6633 seconds, a mean speed of movement of 0.7199 m/s, and a peak speed of 1.52 m/s. In this patient they took longer to perform the total task, but were faster on all of the movements, and moved through a greater range of motion (i.e., the path length of the total movement can be calculated from the image capture device information). In this same patient we the patient perform a bradykinesia test where the patient was asked to perform 10 arm flexion-extension movements. After each flexion or extension movement, the subject is asked to stop. The movements were performed as fast as possible. For their right arm they demonstrated a baseline total time of task of 24.125 seconds, and average movement duration of 0.724 seconds, a mean speed of movement of 0.525 m/s, and a peak speed of 1.20 m/s. Following the 10^{th} simulation they demonstrated a total time of task of 18 seconds, and average movement duration of 0.73 seconds, a mean speed of movement of 0.582 m/s, and a peak speed of 1.27 m/s. We had the patient perform a task where they were asked to combine movements (flexion followed by hand opening/closing followed by extension followed by hand opening/closing) 10 times as fast as possible. For their right arm the patient had a baseline total task time of 32.03 seconds and a total task time of 26.5 seconds following the 10^{th} day of stimulation.

What is claimed is:

1. A motion analysis system comprising:
an image capture device;
at least one external body motion sensor; and
a central processing unit (CPU) with storage coupled to the CPU for storing instructions that when executed by the CPU cause the CPU to:
receive a first set of motion data from the image capture device related to at least one joint of a subject while the subject is performing a task;
receive a second set of motion data from the external body motion sensor related to the at least one joint of the subject while the subject is performing the task;
calculate kinematic and/or kinetic information about the at least one joint of a subject from a combination of the first and second sets of motion data; and
output the kinematic and/or kinetic information for purposes of assessing a movement disorder, wherein the kinematic and/or kinetic information comprises total duration of the task performance; number of movements performed, movement mean speed, movement peak speed, movement duration, movement smoothness, and path length.

2. The motion analysis system according to claim 1, further comprising a force plate.

3. The motion analysis system according to claim 2, wherein the CPU is further caused to:
receive balance data from the force plate; calculate kinematic and/or kinetic information about the at least one joint of a subject from a combination of the first and second sets of motion data and the balance data; and output the kinematic and/or kinetic information for purposes of assessing a movement disorder.

4. The motion analysis system according to claim 2, further comprising a device for eye tracking.

5. The motion analysis system according to claim 4, further comprising a device for voice tracking.

6. The motion analysis system according to claim 1, wherein the task is selected from the group consisting of: discrete flexion of a limb; discrete extension of a limb; continuous flexion of a limb; continuous extension of a limb; flexion of a hand; extension of a hand; walking; and any combination thereof.

7. The motion analysis system according to claim 1, wherein the movement disorder is selected from the group consisting of: Parkinson's disease; Parkinsonism; Dystonia; Cerebral Palsy; Bradykinesia; Chorea; Huntington's Disease; Ataxia; Tremor; Essential Tremor; Myoclonus; tics; Tourette Syndrome; Restless Leg Syndrome; and Stiff Person Syndrome.

8. The motion analysis system according to claim 1, wherein the external body motion sensor is an accelerometer.

9. The motion analysis system according to claim 8, wherein the external body motion sensor further comprises a gyroscope and the second set of motion data further comprises gyroscopic data.

10. The motion analysis suite according to claim 1, wherein the first and second sets of motion data relate to a plurality of joints.

11. The motion analysis suite according to claim 1, wherein the CPU is further caused to render received data from the image capture device as a skeletal joint map.

12. The motion analysis suite according to claim 1, wherein software of the image capture device renders received data as a skeletal joint map and then sends the skeletal joint map to the CPU.
